(19) United States
(12) Patent Application Publication
(10) Pub. No.: US 2025/0145978 A1
(43) Pub. Date: May 8, 2025

(54) GLYCOSYLATED PROTEIN HAVING GLUCOCEREBROSIDASE ACTIVITY

(71) Applicant: NIPPON SHOKUBAI CO., LTD., Osaka-shi, Osaka (JP)

(72) Inventors: Daisuke TATEIWA, Suita-shi, Osaka (JP); Yuki MAKINO, Suita-shi, Osaka (JP); Taiji SHIMODA, Kyoto-shi, Kyoto (JP); Yuji NISHIUCHI, Kyoto-shi, Kyoto (JP); Takefumi MURASE, Kyoto-shi, Kyoto (JP); Yuka MATSUDA, Kyoto-shi, Kyoto (JP)

(21) Appl. No.: 18/833,876

(22) PCT Filed: Jan. 26, 2023
(86) PCT No.: PCT/JP2023/002409

§ 371 Date: Jul. 26, 2024

(30) Foreign Application Priority Data
Jan. 31, 2022 (JP) 2022-013048

(51) Int. Cl. C12N 9/24

(52) U.S. Cl. CPC C12N 9/2402; C12Y 302/01045

(57) ABSTRACT

The present invention provides a glycosylated protein having glucocerebrosidase activity. A glycosylated protein having glucocerebrosidase activity, the glycosylated protein being obtained by adding a sugar chain having a single structure to a protein added with no sugar chain and having glucocerebrosidase activity.

### TECHNICAL FIELD

The present invention relates to a glycosylated protein having glucocerebrosidase activity.

### BACKGROUND ART

Lysosomal disease is a hereditary disease caused by activity decrease or defects in lysosomal enzymes and their related factors and by the resultant storage of the substrates of such enzymes in the living body. For example, in Gaucher disease that is one type of lysosomal disease, activity decrease in glucocerebrosidase (β-glucosidase; GBA) causes storage of glucocerebroside in cells such as macrophages in reticuloendothelial tissues, and as a result, symptoms and observations such as splenohepatomegaly; anemia and decreased platelet count associated with enhancement of splenic function; bone lesions; increase in the levels of blood acidic phosphatase and angiotensin converting enzyme; and the like are observed (Non-Patent Literature 1).

As a method for treating such a lysosomal disease, enzyme replacement therapy has been frequently employed heretofore. For example, in Gaucher disease, a recombinant enzyme expressed by using a cDNA encoding human glucocerebrosidase in a Chinese hamster ovary (CHO) cell strain is used with a sugar chain-altered form so that to be uptaken into target cell macrophages easily (for example, mannose residues are added at the non-reducing terminal of the enzyme in order to facilitate recognition by the mannose receptor present on the surface of target cell macrophages).

However, production of a recombinant enzyme using a mammalian cultured cell such as the above-described CHO cell strain as a host has problems in that a culture solution is expensive, there is a risk of infection by a zoonotic virus, proliferation of cells is slow, and a sugar chain structure is heterogeneous. A technique for producing a recombinant of glucocerebrosidase (GBA) using a cell derived from a plant as a host has also been proposed (Patent Literature 1). However, in a recombinant enzyme produced using a eukaryotic organism such as a plant or a Saccharomyces cerevisiae as a host, the structure of a sugar chain added to the enzyme by post-translational modification is greatly different from that of a mammalian cell, and thus there is a problem in that the recombinant enzyme exhibits antigenicity to a mammal. Therefore, there is a problem in that a recombinant enzyme produced using a cell derived from a wild-type plant or a Saccharomyces cerevisiae as a host is used as a biopharmaceutical drug.

### CITATION LIST

### Patent Literature

Patent Literature 1: JP 2006-524506 A

### Non-Patent Literature

Non-Patent Literature 1: Bou-Gharios G. et al., Histochem J. (1993) 25, 593-605

### SUMMARY OF INVENTION

As described above, a conventional glucocerebrosidase protein to which a sugar chain is added has a problem caused by a sugar chain structure.

Therefore, an object of the present invention is to provide a glycosylated protein having glucocerebrosidase activity.

The present inventors have conducted intensive studies in view of the above problems. As a result, the present inventors have found that the above problems can be solved by a glycosylated protein having glucocerebrosidase activity, the glycosylated protein being obtained by adding a sugar chain having a single structure to a protein added with no sugar chain and having glucocerebrosidase activity, and have completed the present invention.

### DESCRIPTION OF EMBODIMENTS

Hereinafter, embodiments according to the present invention will be described in detail. However, the following descriptions are illustrative for explaining the present invention, and are not intended to limit the technical scope of the present invention to this description range only.

In the present specification, the phrase “X to Y” indicating a range includes X and Y and means “X or more and Y or less”. Unless otherwise specified, operations and measurements of physical properties and the like are measured under the conditions of room temperature (20 to 25° C.)/relative humidity of 40 to 50% RH.

One aspect of the present invention is a glycosylated protein having glucocerebrosidase activity, the glycosylated protein being obtained by adding a sugar chain having a single structure to a protein added with no sugar chain and having glucocerebrosidase activity. According to the present aspect, there is provided a glycosylated protein having a sugar chain having a single structure and having glucocerebrosidase activity.

In the present specification, the “glucocerebrosidase activity” means an activity of hydrolyzing glucocerebroside. The presence or absence of glucocerebrosidase activity is determined based on the presence or absence of enzyme reactivity with a synthetic substrate (p-nitrophenyl-β-D-glucopyranoside) described in the section of EXAMPLES described later. The specific activity of the protein added with no sugar chain and having glucocerebrosidase activity after a refolding treatment according to the present invention is, for example, 0.5 U/mg or more, preferably 0.6 U/mg or more, and more preferably 1.2 U/mg or more. The glycosylated protein of the present invention has a specific activity of 80% or more, preferably 100% or more, with respect to the specific activity of the protein added with no sugar chain and having glucocerebrosidase activity.

The mature protein of glucocerebrosidase is a polypeptide consisting of 497 amino acid residues generated by cleavage of a propeptide from a precursor protein consisting of 536 amino acid residues. Examples of biopharmaceuticals of glucocerebrosidase put on the market with Gaucher disease as an indication include Cerezyme (registered trademark) (produced from Chinese hamster ovary (CHO) cells), VPRIV (registered trademark) (produced from human fibrosarcoma cells (HT1080)), and Elelyso (registered trademark) (produced from plant (carrot) cells).

Examples of the amino acid sequence constituting the protein added with no sugar chain and having glucocerebrosidase activity according to the present invention include an amino acid sequence constituting a human wild-type GBA protein; an amino acid sequence constituting Cerezyme (registered trademark), VPRIV (registered trademark), Elelyso (registered trademark), or the like; and an amino acid sequence having identity (synonymous with “homology” in the present specification) of 90% or more (more preferably 95% or more, still more preferably 99% or more) therewith.

In the present specification, the identity of the amino acid sequence can be determined using an analysis program such as BLAST, FASTA, or CLUSTAL W. In the case of using BLAST, a default parameter of the program is used.

Here, the “identity” of the amino acid sequence is expressed in percentage as follows: two amino acid sequences to be compared are arranged in parallel such that the amino acid residues of both the amino acid sequences match as many times as possible, and then the number of matched amino acid residues is divided by the total number of amino acid residues. In the alignment, a gap is appropriately inserted into one or both of the two sequences to be compared as necessary, and one inserted gap is counted as one amino acid residue to determine the total number of amino acid residues. When the total number of amino acid residues thus determined is different from between the two sequences to be compared, the sequence identity (%) is calculated by dividing the number of matched amino acid residues by the total number of amino acid residues of the longer sequence.

In one embodiment of the present invention, the protein added with no sugar chain and having glucocerebrosidase activity contains an amino acid sequence set forth in SEQ ID NO: 1 or 2 or an amino acid sequence having identity of 90% or more therewith.

The amino acid sequence set forth in SEQ ID NO: 1 corresponds to the amino acid sequence of Cerezyme (the amino acid at a position corresponding to position 495 is histidine (H) unlike a human wild-type GBA protein). The amino acid sequence is shown below, and a base sequence (including a termination codon) of a gene (cDNA) encoding the amino acid sequence is shown in SEQ ID NO: 134. In the present specification, a gene encoding the amino acid sequence set forth in SEQ ID NO: 1 is also simply referred to as “GBA gene”.

[Chemical Formula 1]
(SEQ ID NO: 1)
ARPCIPKSFGYSSVVCVCNATYCDSFDPPTFPALGTFSRYESTRS
GRRMELSMGPIQANHTGTGLLLTLQPEQKFQKVKGFGGAMTDAAA
LNILALSPPAQNLLLKSYFSEEGIGYNIIRVPMASCDFSIRTYTY
ADTPDDFQLHNFSLPEEDTKLKIPLIHRALQLAQRPVSLLASPWT
SPTWLKTNGAVNGKGSLKGQPGDIYHQTWARYFVKFLDAYAEHKL
QFWAVTAENEPSAGLLSGYPFQCLGFTPEHQRDFIARDLGPTLAN
STHHNVRLLMLDDQRLLLPHWAKVVLTDPEAAKYVHGIAVHWYLD
FLAPAKATLGETHRLFPNTMLFASEACVGSKFWEQSVRLGSWDRG
MQYSHSIITNLLYHVVGWTDWNLALNPEGGPNWVRNFVDSPIIVD
ITKDTFYKQPMFYHLGHFSKFIPEGSQRVGLVASQKNDLDAVALM
HPDGSAVVVVLNRSSKDVPLTIKDPAVGFLETISPGYSIHTYLWH
RQ

The amino acid sequence set forth in SEQ ID NO: 2 corresponds to the amino acid sequence of VPRIV (the amino acid at a position corresponding to position 495 is arginine (R) unlike a human wild-type GBA protein). The amino acid sequence is shown below.

[Chemical Formula 2]
(SEQ ID NO: 2)
ARPCIPKSFGYSSVVCVCNATYCDSFDPPTFPALGTFSRYESTRS
GRRMELSMGPIQANHTGTGLLLTLQPEQKFQKVKGFGGAMTDAAA
LNILALSPPAQNLLLKSYFSEEGIGYNIIRVPMASCDFSIRTYTY
ADTPDDFQLHNFSLPEEDTKLKIPLIHRALQLAQRPVSLLASPWT
SPTWLKTNGAVNGKGSLKGQPGDIYHQTWARYFVKFLDAYAEHKL
QFWAVTAENEPSAGLLSGYPFQCLGFTPEHQRDFIARDLGPTLAN
STHHNVRLLMLDDQRLLLPHWAKVVLTDPEAAKYVHGIAVHWYLD
FLAPAKATLGETHRLFPNTMLFASEACVGSKFWEQSVRLGSWDRG
MQYSHSIITNLLYHVVGWTDWNLALNPEGGPNWVRNFVDSPIIVD
ITKDTFYKQPMFYHLGHFSKFIPEGSQRVGLVASQKNDLDAVALM
HPDGSAVVVVLNRSSKDVPLTIKDPAVGFLETISPGYSIHTYLWR
RQ

In a preferred embodiment of the present invention, from the viewpoint of ease of sugar chain modification and glucocerebrosidase activity, the protein added with no sugar chain and having glucocerebrosidase activity is a protein containing at least one of the following amino acid substitutions in an amino acid sequence set forth in SEQ ID NO: 1 or 2:
(1) a substitution of an amino acid at a position corresponding to position 61 of SEQ ID NO: 1 or 2 with cysteine (T61C);
(2) a substitution of an amino acid at a position corresponding to position 98 of SEQ ID NO: 1 or 2 with cysteine (P98C);
(3) a substitution of an amino acid at a position corresponding to position 143 of SEQ ID NO: 1 or 2 with cysteine (Q143C);
(4) a substitution of an amino acid at a position corresponding to position 224 of SEQ ID NO: 1 or 2 with cysteine (K224C);
(5) a substitution of an amino acid at a position corresponding to position 321 of SEQ ID NO: 1 or 2 with cysteine (K321C); and
(6) a substitution of an amino acid at a position corresponding to position 407 of SEQ ID NO: 1 or 2 with cysteine (T407C).

Examples of the amino acid sequence having at least one of the above-described amino acid substitutions include amino acid sequences set forth in SEQ ID NOs: 16, 24, 28, 30, 37, 39, 41, 43 to 49, 136, 137, and 141 to 145.

Still more preferably, the protein added with no sugar chain and having glucocerebrosidase activity is a protein according to the present invention includes at least one selected from amino acid sequences set forth in SEQ ID NOS: 24, 30, 43, 136, 137, and 141 to 145.

In a preferred embodiment of the present invention, from the viewpoint that glucocerebrosidase activity can be further improved, the protein added with no sugar chain and having glucocerebrosidase activity is a protein containing at least one of the following amino acid substitutions in an amino acid sequence set forth in SEQ ID NO: 1 or 2:
(7) a substitution of an amino acid at a position corresponding to position 26 of SEQ ID NO: 1 or 2 with leucine (F26L);
(8) a substitution of an amino acid at a position corresponding to position 26 of SEQ ID NO: 1 or 2 with isoleucine (F26I);
(9) a substitution of an amino acid at a position corresponding to position 126 of SEQ ID NO: 1 or 2 with threonine (C126T);
(10) a substitution of an amino acid at a position corresponding to position 126 of SEQ ID NO: 1 or 2 with serine (C126S) and a substitution of an amino acid at a position corresponding to position 342 of SEQ ID NO: 1 or 2 with serine (C342S);
(11) a substitution of an amino acid at a position corresponding to position 57 of SEQ ID NO: 1 or 2 with cysteine (Q57C);
(12) a substitution of an amino acid at a position corresponding to position 60 of SEQ ID NO: 1 or 2 with cysteine (H60C);
(13) a substitution of an amino acid at a position corresponding to position 63 of SEQ ID NO: 1 or 2 with cysteine (T63C);
(14) a substitution of an amino acid at a position corresponding to position 143 of SEQ ID NO: 1 or 2 with cysteine (Q143C);
(15) a substitution of an amino acid at a position corresponding to position 145 of SEQ ID NO: 1 or 2 with cysteine (H145C);
(16) a substitution of an amino acid at a position corresponding to position 224 of SEQ ID NO: 1 or 2 with cysteine (K224C); and
(17) a substitution of an amino acid at a position corresponding to position 321 of SEQ ID NO: 1 or 2 with cysteine (K321C).

The protein added with no sugar chain and having glucocerebrosidase activity according to the present invention is more preferably a protein containing an amino acid sequence having at least one of the following amino acid substitutions in an amino acid sequence of SEQ ID NO: 1 or 2:
(a-1) (7) F26L;
(a-2) (9) C126T;
(a-3) (8) F26I and (9) C126T;
(a-4) (7) F26L and (9) C126T;
(a-5) (10) C126S and C342S;
(a-6) (9) C126T and (11) Q57C;
(a-7) (9) C126T and (12) H60C;
(a-8) (9) C126T and (13) T63C;
(a-9) (9) C126T and (14) Q143C;
(a-10) (9) C126T and (15) H145C;
(a-11) (9) C126T and (16) K224C; and
(a-12) (9) C126T and (17) K321C.

Provided that, in (a-1) to (a-12), amino acids at the following positions are not substituted:
in (a-2), an amino acid at a position corresponding to position 142;
in (a-2), an amino acid at a position corresponding to position 144;
in (a-2), an amino acid at a position corresponding to position 147;
in (a-2), an amino acid at a position corresponding to position 171;
in (a-2), an amino acid at a position corresponding to position 347;
in (a-2), an amino acid at a position corresponding to position 407;
in (a-4), an amino acid at a position corresponding to position 248;
in (a-9), an amino acid at a position corresponding to position 77;
in (a-9), an amino acid at a position corresponding to position 290;
in (a-9), an amino acid at a position corresponding to position 293;
in (a-9), an amino acid at a position corresponding to position 333; and
in (a-9), an amino acid at a position corresponding to position 466.

Examples of the amino acid sequence having at least one of the above-described amino acid substitutions include amino acid sequences set forth in SEQ ID NOs: 3 to 5, 7, 9 to 30, 32, 33, 35, 37 to 39, 41 to 51, 136, 137, and 141 to 145.

Still more preferably, the protein according to the present invention contains at least one selected from amino acid sequences set forth in SEQ ID NOS: 3, 5, 7, 9, 10, 12 to 16, 18 to 28, 30, 32, 33, 35, 37 to 39, 42, 47, 136, 137, and 141 to 145.

In a preferred embodiment of the present invention, from the viewpoint that stability can be further improved, the protein added with no sugar chain and having glucocerebrosidase activity according to the present invention is a protein containing at least one of the following amino acid substitutions in an amino acid sequence of SEQ ID NO: 1 or 2:
(18) a substitution of an amino acid at a position corresponding to position 248 of SEQ ID NO: 1 or 2 with serine (C248S) and a substitution of an amino acid at a position corresponding to position 342 of SEQ ID NO: 1 or 2 with serine (C342S);
(19) a substitution of an amino acid at a position corresponding to position 126 of SEQ ID NO: 1 or 2 with threonine (C126T) and a substitution of an amino acid at a position corresponding to position 342 of SEQ ID NO: 1 or 2 with serine (C342S);
(20) a substitution of an amino acid at a position corresponding to position 126 of SEQ ID NO: 1 or 2 with serine (C126S), a substitution of an amino acid at a position corresponding to position 248 of SEQ ID NO: 1 or 2 with serine (C248S), and a substitution of an amino acid at a position corresponding to position 342 of SEQ ID NO: 1 or 2 with serine (C342S); and
(21) a substitution of an amino acid at a position corresponding to position 126 of SEQ ID NO: 1 or 2 with threonine (C126T), a substitution of an amino acid at a position corresponding to position 248 of SEQ ID NO: 1 or 2 with serine (C248S), and a substitution of an amino acid at a position corresponding to position 342 of SEQ ID NO: 1 or 2 with serine (C342S).

More desirably, the protein added with no sugar chain and having glucocerebrosidase activity according to the present invention is a protein containing at least one selected from amino acid sequences set forth in SEQ ID NOS: 14, 17, 18, and 51.

The protein added with no sugar chain and having glucocerebrosidase activity according to the present invention may be a protein consisting of the amino acid sequence described above.

The glycosylated protein of the present invention is obtained by adding a sugar chain having a single structure to a protein added with no sugar chain and having glucocerebrosidase activity.

In the glycosylated protein of the present invention, the number of amino acids to which a sugar chain is added may be 1 or more, and is preferably 1 to 4.

In the present specification, the “sugar chain” means a compound in which one or more unit sugars (monosaccharide and a derivative thereof) are linked. When two or more unit sugars are linked, the unit sugars are bonded to each other by dehydration condensation by a glycoside bond. Examples of the saccharides include monosaccharides and polysaccharides contained in a living body (glucose, galactose, mannose, fucose, xylose, N-acetylglucosamine, N-acetylgalactosamine, sialic acid, complexes thereof, and derivatives thereof); degraded polysaccharide; sugar chains degraded or induced from complex biological materials such as glycoproteins, proteoglycans, glycosaminoglycans, and glycolipids; and the like.

In the present specification, the “sugar chain having a single structure” means that, when sugar chains to be added are compared with each other, the types, binding orders, and binding modes of the sugars constituting the sugar chains are the same in the glycosylated protein. The glycosylated protein of the present invention may be one in which only one sugar chain is added per site, but may also include one in which two or three or more sugar chains are added.

The sugar chain according to the present invention is not particularly limited as long as it is a sugar chain that does not lose the glucocerebrosidase activity of the glycosylated protein. The sugar chain may be a sugar chain that exists in vivo as a glycoconjugate (glycopeptide or glycoprotein, proteoglycan, glycolipid, or the like), or may be a sugar chain that does not exist in vivo as a glycoconjugate. Examples of the sugar chain present as a glycoconjugate in vivo include an N-linked sugar chain, O-linked sugar chain, and the like.

In one embodiment of the present invention, the glycosylated protein has at least one sugar chain having a single structure added, and the sugar chain having a single structure has a structure derived from a compound represented by the following Formula 1.

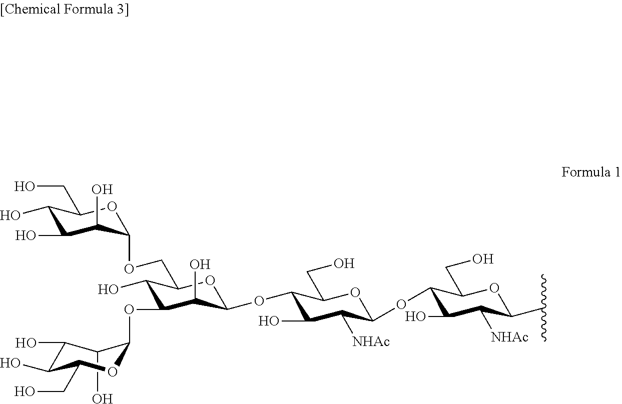

The sugar chain according to the present invention may be a sugar chain composed of the compound represented by the above-described Formula 1, or may be a sugar chain in which another sugar chain structure is bonded to the compound represented by Formula 1. Examples of the sugar chain in which another sugar chain structure is bonded include a high mannose type, a complex type, a hybrid type, and the like.

In one embodiment, the sugar chain according to the present invention consists of the compound represented by the above-described Formula 1.

In the glycosylated protein of the present invention, the amino acid to which a sugar chain is added is not particularly limited, and any amino acid can be used.

The sugar chain may be directly bonded to an amino acid or may be added via a linker.

In one embodiment, the sugar chain is added via a linker. When the sugar chain and an amino acid are bonded via a linker, from the viewpoint of ease of bonding with the linker, examples of the amino acid to which the sugar chain is added include cysteine, aspartic acid, glutamic acid, lysine, arginine, histidine, tryptophan, serine, threonine, tyrosine, asparagine, glutamine, and the like. From the viewpoint of reactivity, the amino acid to which the sugar chain is added is preferably an amino acid selected from the group consisting of cysteine, asparagine, aspartic acid, glutamic acid, lysine, arginine, serine, threonine, and glutamine, and more preferably cysteine.

The linker is not particularly limited, and a conventionally known linker can be used. Examples of the linker include a substituted or unsubstituted alkylene group having 1 to 20 carbon atoms, a substituted or unsubstituted alkenylene group having 2 to 20 carbon atoms, a substituted or unsubstituted alkynylene group having 2 to 20 carbon atoms, a substituted or unsubstituted cycloalkylene group having 3 to 20 carbon atoms, a substituted or unsubstituted cycloalkenylene group having 3 to 20 carbon atoms, a substituted or unsubstituted arylene group having 6 to 20 carbon atoms, a substituted or unsubstituted heteroarylene group having 3 to 20 carbon atoms, —NH—, —O—, —S—, —C(═O)—NH—, —NH—C(═O)—, —O—, —C(═O)—O—, —O—C(═O)—, —S—, —C(═O)—, a polyoxyalkylene group, an amino acid residue, a peptide chain, a polyethylene glycol, a reactive functional group (for example, a group derived from a maleimide structure, a halogenated acetyl structure, an acryloyl structure, or S-Npys (S-3-nitro-2-pyridinesulphenyl), and combinations thereof.

When the sugar chain and an amino acid are bonded via a linker, the number of sugar chains bonded to the linker (the number of sugar chains added to one amino acid) is, for example, 1 or more, and preferably 1 to 3, more preferably 2 or 3, and still more preferably 3 from the viewpoint that the uptake amount into cells can be improved. Examples of a form in which three sugar chains are bonded to the linker include a form in which a sugar chain is bonded to each amino acid of a linker containing a tripeptide.

In a preferred embodiment, in the glycosylated protein of the present invention, the sugar chain having a single structure is added to a cysteine residue constituting the protein added with no sugar chain and having glucocerebrosidase activity. When a sugar chain is added to a cysteine residue, a linker has a reactive functional group, preferably a maleimide structure, at its terminal end.

When the sugar chain is added to a cysteine residue, the protein added with no sugar chain and having glucocerebrosidase activity is preferably a protein containing at least one of the above amino acid substitutions (1) to (6) in an amino acid sequence set forth in SEQ ID NO: 1 or 2.

An example of a method for producing the glycosylated protein of the present invention will be described below.

A method for producing a peptide chain as a raw material of the protein added with no sugar chain and having glucocerebrosidase activity according to the present invention is not particularly limited as long as a sugar chain is not added to a peptide chain, and the peptide chain may be a peptide chain produced by a prokaryote or a peptide chain synthesized by organic synthesis. In the protein according to the present invention, from the viewpoint of high productivity and low cost, preferably, a peptide chain produced by a prokaryote can be used as a raw material.

Examples of the prokaryote include bacteria belonging to the genus Escherichia such as Escherichia coli, the genus Bacillus such as Bacillus subtilis, the genus Pseudomonas such as Pseudomonas putida, and the genus Rhizobium such as Rhizobium meliloti. The prokaryote used in the present invention is preferably E. coli.

In one embodiment, a method for producing the protein added with no sugar chain and having glucocerebrosidase activity according to the present invention includes introducing a vector containing a nucleic acid encoding the protein into a prokaryote to cause the prokaryote to produce a protein raw material, and subjecting the protein raw material which is collected to a folding treatment.

First, the vector containing a nucleic acid encoding the protein according to the present invention is introduced into a prokaryote, and the prokaryote is caused to produce a protein raw material. Thereby, a protein raw material added with no sugar chain can be obtained.

Methods for producing a nucleic acid encoding the protein added with no sugar chain and having glucocerebrosidase activity according to the present invention and a vector containing the nucleic acid are not particularly limited, and conventionally known methods can be used.

As the vector, a known vector, for example, a T vector such as pTAKN-2, or a plasmid vector such as PET-21b(+) can be used.

A method for introducing a vector into a prokaryote is not particularly limited, and a conventionally known method can be appropriately used. Examples of the introduction method include a competent cell method, a conjugate transfer method, a calcium phosphate method, a lipofection method, an electroporation method, and the like.

By culturing the prokaryote into which the vector has been introduced, the prokaryote can be caused to produce a protein raw material. Culturing the prokaryote can be carried out according to the usual method used for a selected prokaryote.

Depending on the type of prokaryotes to be used, a prokaryote is cultured under aerobic or anaerobic conditions. In the former case, the prokaryote may be cultured by shaking, aeration stirring, or the like. The culture conditions (culture temperature, culture time, pH of medium, and the like) are appropriately selected depending on the composition of a medium and a culture method, and are not particularly limited as long as the prokaryote can grow, and can be appropriately selected according to the type of prokaryote to be cultured.

Since the protein added with no sugar chain and having glucocerebrosidase activity according to the present invention is not added with a sugar chain by post-translational modification, that is, it is desired that the protein is not subjected to post-translational modification.

A method for collecting a protein raw material produced by a prokaryote is not particularly limited, and a conventionally known method can be appropriately used. For example, when the protein raw material is present inside a prokaryote, the prokaryote is collected from the obtained culture by a method such as centrifugation or filtration, and the collected prokaryote is disrupted by a mechanical method using beads or the like or an enzymatic method. After crushing, the insoluble fraction is collected and treated with a buffer containing a surfactant, whereby the protein raw material can be collected.

Next, the collected protein raw material is subjected to a folding treatment (may be a refolding treatment including a denaturation treatment performed in advance).

The folding treatment can be performed, for example, by adding a buffer containing an oxidizing agent and a reducing agent (oxidized glutathione/reduced glutathione, cystine/cysteine, cysteamine/cystamine, or the like) to a liquid containing the collected protein raw material and allowing the mixture to stand still at about 20° C. to about 30° C. for about 1 day to 7 days. An additive such as sucrose or glycerol can be further added to the buffer.

The collected protein raw material may be subjected to a denaturation (solubilization) treatment as a necessary before the folding treatment. The denaturation treatment can be performed using a denaturant such as 6 M guanidine hydrochloride or 8 M urea. By performing the denaturation treatment, the collected protein raw material can be brought into an unfolded state.

In this way, a protein added with no sugar chain and having glucocerebrosidase activity can be obtained.

A method for producing the sugar chain having a single structure according to the present invention is not particularly limited, and a conventionally known method can be appropriately used. For example, the methods described in WO 03/008431 A1, WO 2004/058984 A1, WO 2004/058824 A1, WO 2004/070046 A1, WO 2007/011055 A1, and the like can be used.

As a method for adding a sugar chain having a single structure to a protein added with no sugar chain and having glucocerebrosidase activity, for example, the method described in WO 2014/157107 A1 or the like can be used.

Hereinafter, as an example, a method for adding a sugar chain to cysteine constituting the protein added with no sugar chain and having glucocerebrosidase activity by chemical synthesis will be described. As the present method, the method described in WO 2005/010053 A1 or the like can be used.

When the sugar chain is bonded to cysteine via a linker, a sugar chain derivative (sugar chain modification reagent in EXAMPLES) in which the sugar chain and the linker are bonded is reacted with the protein added with no sugar chain and having glucocerebrosidase activity. The sugar chain derivative is, for example, a compound in which a group bonded to the carbon at position 1 of GalNac of the sugar chain terminal is substituted with a linker in the compound represented by Formula 1. The methods for producing the linker and the sugar chain derivative are not particularly limited, and conventionally known methods can be used.

Specifically, the sugar chain derivative and the protein added with no sugar chain and having glucocerebrosidase activity are reacted in a phosphate buffer at about 0° C. to room temperature. The phosphate buffer may contain tris(2-carboxyethyl)phosphine hydrochloride (TCEP) or the like in order to prevent the formation of a dimer. The final concentration of TCEP or the like is, for example, 10 μM to 10 mM.

After completion of the reaction, a glycosylated protein can be obtained by purification by HPLC.

The glycosylated protein having glucocerebrosidase activity of the present invention is different from a conventional sugar chain-modified glucocerebrosidase protein and is added with a sugar chain having a single structure. Therefore, stable drug efficacy can be exhibited. Stability at a pH near neutrality can be improved (see EXAMPLES). Therefore, the glycosylated protein can be suitably used in the treatment of lysosomal diseases such as Gaucher disease.

Therefore, one embodiment of the present invention relates to a composition containing the glycosylated protein described above, preferably a composition containing only one kind of the glycosylated protein described above. By using the glycosylated protein of the present invention, the kind and ratio of the glycosylated protein in the composition can be controlled. For example, the fact that there is only one kind of the glycosylated protein in the composition means that there are glycosylated proteins in the composition, in which the position of an amino acid to which a sugar chain is added, the number of amino acids to which a sugar chain is added, and the number of sugar chains to be added is the same.

The composition according to the present invention can have a constant quality, and thus is particularly suitable for use applications such as pharmaceuticals and assays.

### EXAMPLES

Hereinafter, the present invention will be specifically described with reference to Examples, but the present invention is not limited thereto. In particular, it is possible to appropriately use other generally known means for portions (1-1 to 2-2) related to the construction and culture of a bacterial strain producing a GBA protein and a recombinant GBA protein and the disruption of cells.

In Examples, the plasmid number and the recombinant protein number are given the same number.

[Construction of GBA Gene and Altered Gene-Introduced Recombinant E. coli Thereof]

### 1-1. Synthesis of Glucocerebrosidase (GBA) Gene

A GBA gene represented by SEQ ID NO: 135 is obtained by adding an initiation codon (atg) to the 5′-end of a codon encoding a mature GBA protein from which a signal peptide has been removed, and by making a change so as to obtain a sequence optimized for codon usage frequency of E. coli (E. coli K-12 strain), The synthesis of the GBA gene represented by SEQ ID NO: 135 was outsourced to Eurofins Genomics K.K., and delivered in a state of being inserted into pTAKN-2 containing an ampicillin resistance gene.

1-2. Preparation of Plasmid into which GBA Gene is Inserted

For expression studies in E. coli, the GBA gene obtained above was subcloned between the NdeI site and the His tag of the pET-21b(+) plasmid vector (Novagen). Specifically, PCR using either pET-21b(+) or pTAKN-2 into which the GBA gene was inserted as a template was performed to obtain an amplification product of linearized pET-21b(+) and the GBA gene (excluding a termination codon).

TABLE 1
Sequence of primer

| | |
|---|---|
| For pET-21b(+) amplification | |
| Forward primer | 5′-CAGTAAGGATCCGA |
| (SEQ ID NO: 130) | ATTCGAGCTCCG-3′ |
| Reverse primer | 5′-TGATGATGATGGCC |
| (SEQ ID NO: 131) | CATGGTATATCTCCTTC |
| | TTAAAGTTAAACAAAAT |
| | TATTTCTAGAG-3′ |
| For pTAKN-2 amplification | |
| Forward primer | 5′-GGGCCATCATCATC |
| (SEQ ID NO: 132) | ATCATCATCATCATCAT |
| | CACAGCAGCGGCCATAT |
| | CGACGACGACGACAAGG |
| | CGCGCCCATGTATCC |
| | C-3′ |
| Reverse primer | 5′-TTCGGATCCTTACT |
| (SEQ ID NO: 133) | GACGATGCGACAGATAG |
| | GTG-3′ |

The PCR amplification product obtained above was subjected to a treatment (cleavage by the restriction enzyme DpnI and ligation) using In-Fusion HD Cloning Kit (Takara Bio Inc.) to obtain a pET-21b(+) plasmid vector into which the GBA gene was inserted (referred to as “H495 type” in the present specification). The GBA gene inserted into the plasmid vector encodes the amino acid sequence set forth in SEQ ID NO: 1.

1-3. Preparation of Plasmid into which Altered GBA Gene is Inserted

PCR using a plasmid into which the GBA gene prepared in the above 1-2. was inserted as a template was performed using a primer for mutation introduction (intended to substitute the amino acid encoded by the GBA gene with another amino acid) described in Table 1 below to variously amplify a plasmid (linearized plasmid) in which a mutation was introduced into the GBA gene (excluding the termination codon). Substitution sites of the amino acid sequence and codons corresponding to the substituted amino acids in the various altered GBA genes are as shown in Table 2. According to the manual, the obtained PCR amplification product (linearized plasmid) was self-ligated and circularized with T4 Polynucleotide Kinase (TOYOBO Co., Ltd.) and Ligation high Ver. 2 (TOYOBO Co., Ltd.) to obtain a plasmid into which the altered GBA gene was inserted (Table 5). When a plurality of mutations were introduced, mutations were added by repeating the same method as described above.

TABLE 1

| SEQ ID | Altered | | |
|---|---|---|---|
| NO: | site | | |
| 52 | F26L | Forward primer | 5′-ctgGATCCGCCCACTTTTCCC-3′ |
| 53 | | Reverse primer | 5′-CGAATCGCAGTACGTGGC-3′ |
| 54 | C126S | Forward primer | 5′-agcGACTTTTCGATTCGCACCTATAC-3′ |
| 55 | | Reverse primer | 5′-CGAGGCCATTGGAACGCG-3′ |
| 56 | C126T | Forward primer | 5′-CGAGGCCATTGGAACGCG-3′ |
| 57 | | Reverse primer | 5′-ggtCGAGGCCATTGGAACGCG-3′ |
| 58 | C342G | Forward primer | 5′-GTGGGGAGCAAGTTTTGGGAG-3′ |
| 59 | | Reverse primer | 5′-gccTGGTTCGCTAGCAAAGAGCATC-3′ |
| 60 | C342S | Forward primer | 5′-AAGGAagcGTGGGGAGCAAGTTTTGG-3′ |
| 61 | | Reverse primer | 5′-CCCACgctTGCTTCGGTAGCAAAGAGC-3′ |
| 62 | C342T | Forward primer | 5′-AAGCAcccGTGGGGAGCAAGTTTTGG-3′ |
| 63 | | Reverse primer | 5′-CCCACggtTGCTTCGCTAGCAAAGAGC-3′ |
| 64 | F26I | Forward primer | 5′-attGATCCGCCCACTTTTCCC-3′ |
| 65 | | Reverse primer | 5′-GGAATCGAGTACGTGGC-3′ |
| 66 | C248S | Forward primer | 5′-agcCTGGGTTTTACGCCGGAAC-3′ |
| 67 | | Reverse primer | 5′-TTGAAACGGATAACCCGAGAG-3′ |
| 68 | S184C | Forward primer | 5′-tgcTTACCGGAAGAGGATACGAAAC-3′ |
| 69 | | Reverse primer | 5′-GAAATTGTGCAGCTGAAAGTCATC-3′ |
| 70 | T61C | Forward primer | 5′-tgcGGTACTGGGCTGTTGCTTAC-3′ |
| 71 | | Reverse primer | 5′-GTGATTGGCCTGAATAGGGC-3′ |
| 72 | I56C | Forward primer | 5′-CAGGCCAATCACACGGTAC-3′ |
| 73 | | Reverse primer | 5′-gcaAGGGCCGATAGACAGTTCC-3′ |
| 74 | [symbol] | Forward primer | 5′-GCCAATCACACCGGTACTG-3′ |
| 75 | | Reverse primer | 5′-gcaAATAGGGCCCATAGACAGTTC-3′ |
| 76 | A58C | Forward primer | 5′-AATCACACCGGTACTGGG-3′ |
| 77 | | Reverse primer | 5′-gcaCTGAATAGGGCCCATAGAC-3′ |
| 78 | N59C | Forward primer | 5′-AGGGCTGCtgcCACACCGGTACTGGGCTG-3′ |
| 79 | | Reverse primer | 5′-GAGAAgcaGTGCAGCTGAAAGTCATC-3′ |
| 80 | [symbol] | Forward primer | 5′-ACCGGTACTGGGCTGTTG-3′ |
| 81 | | Reverse primer | 5′-gcaATTGGCCTGAATAGGGCG-3′ |
| 82 | T61C | Forward primer | 5′-tgcGGTACTGGGCTGTTGCTTAC-3′ |
| 83 | | Reverse primer | 5′-GTGATTGGCCTGAATAGGGC-3′ |
| 84 | Q62C | Forward primer | 5′-ACTGGGCTGTTGCTTACC-3′ |
| 85 | | Reverse primer | 5′-gcaGGTGTGATTGGCCTGAATAG-3′ |
| 86 | T63C | Forward primer | 5′-GGGCTGTTGCTTACCCTC-3′ |
| 87 | | Reverse primer | 5′-gcaACGGGTGTGATTGGCCTG-3′ |
| 88 | G64C | Forward primer | 5′-CTGTTGCTTACCCTCCAAC-3′ |
| 89 | | Reverse primer | 5′-gcaAGTACCGGTGTGATTGGC-3′ |
| 90 | P98C | Forward primer | 5′-tgcCTGGGTTTTACGCCGGAAC-3′ |
| 91 | | Reverse primer | 5′-TTGAAACGGATAACCCGAGAG-3′ |
| 92 | F142 [symbol] | Forward primer | 5′-CAGCTGCACAATTTCTCGTTAC-3′ |
| 93 | | Reverse primer | 5′-gcaGTCATCCGGCGTATCCGC-3′ |
| 94 | Q143C | Forward primer | 5′-CTGCACAATTTCTCGTTACC-3′ |
| 95 | | Reverse primer | 5′-gcaAAAGTCATCCGGCGTATC-3′ |
| 96 | L144C | Forward primer | 5′-CACAATTTCTCTCGTTAGCGGAAG-3′ |
| 97 | | Reverse primer | 5′-gcaCTGAAAGTCATCCGGCGTATC-3′ |
| 98 | H145C | Forward primer | 5′-AATTTCTGTGGTTAGCGGAAGAGG-3′ |
| 99 | | Reverse primer | 5′-gcaCAGCTGAAAGTCATCCGGC-3′ |
| 100 | [symbol] 146 [symbol] | Forward primer | 5′-TGCACtgcTTCTCGTTACCGGAAGAG-3′ |
| 101 | | Reverse primer | 5′-GTGTGgcaGGCCTGAATAGGGCCCATAG-3′ |
| 102 | F147C | Forward primer | 5′-TCGTTACCGGAAGAGGATACG-3′ |
| 103 | | Reverse primer | 5′-gcaATTGTGCAGCTGAAAGTCATC-3′ |
| 104 | S148C | Forward primer | 5′-tgcTTACCGGAAGAGGATACGAAAC-3′ |
| 105 | | Reverse primer | 5′-GAAATTGTGCAGCTGAAAGTCATC-3′ |
| 106 | P171C | Forward primer | 5′-GTGTCCCTGCTGGCTTCAC-3′ |
| 107 | | Reverse primer | 5′-gcaACGCTGTCGGAATTGTAAC-3′ |
| 108 | K22 [symbol] C | Forward primer | 5′-CTGCAGTTTTGGGCCGTG-3′ |
| 109 | | Reverse primer | 5′-gcaATGCTCAGCATACGCATC-3′ |
| 110 | P299C | Forward primer | 5′-GAAGCTGCGAAATATGTG-3′ |
| 111 | | Reverse primer | 5′-gcaATCGGTTAACACCACTTTG-3′ |
| 112 | K321C | Forward primer | 5′-GCCACCTTGGGAGAAACTC-3′ |
| 113 | | Reverse primes | 5′-gcaGGGAGGCGCCAGAAAATC-3′ |
| 114 | F347C | Forward primer | 5′-cTGGGAGCAAAGCGTCCG-3′ |
| 115 | | Reverse primer | 5′-caCTTGCTGGCGACgctTG-3′ |
| 116 | T407C | Forward primer | 5′-AAGGATACGTTCTACAAACAGC-3′ |
| 117 | | Reverse primer | 5′-gcaGATATCGACAATGATCGGAGAATC-3′ |
| 118 | K441C | Forward primer | 5′-AACGACCTAGATGCAGTAGC-3′ |
| 119 | | Reverse primer | 5′-gcaCTGGGAGGCTACCAGGCC-3′ |
| 120 | K77C | Forward primer | 5′-GTGAAAGGCTTTGGCGGC-3′ |
| 121 | | Reverse primer | 5′-gcaCTGAAATTTCTGCTCCGGTTGG-3′ |
| 122 | H29 [symbol] C | Forward primer | 5′-TGGGCCAAAGTGGTGTTAAC-3′ |
| 123 | | Reverse primer | 5′-gcaCGGAAGGAGCAACCGTTG-3′ |
| 124 | T297C | Forward primer | 5′-GATCCAGAAGCTGCGAAATATG-3′ |
| 125 | | Reverse primer | 5′-gcaTAACACCACTTTGGCCCAAT-3′ |
| 126 | N333C | Forward primer | 5′-ACGATGCTCTTTGCTAGCGA-3′ |
| 127 | | Reverse primer | 5′-gcaCGGGAAAGAGGCGATGAGTTTC-3′ |
| 128 | K46 [symbol] C | Forward primer | 5′-GACGTACCGCTGACCATC-3′ |
| 129 | | Reverse primer | 5′-gcaTGAACTGCGGTTTAAAACAAC-3′ |
| 146 | S242C | Forward primer | 5′-tgcGGTTATCCGTTTCAATGCCTG-3′ |
| 147 | | Reverse primer | 5′-GAGCAAACCTGCACTTGGC-3′ |

[symbol]

indicates data missing or illegible when filed

TABLE 5
(Table 2)

| Amino acid residue | |
|---|---|
| after substitution | Codon |
| R | cgc |
| S | agc |
| T | acc |
| C | tgc |
| G | ggc |
| I | att |
| L | ctg |

1-4. Construction of Recombinant E. coli Strain

Each of the plasmids constructed in 1-2 and 1-3 was transformed into a competent cell of E. coli (ECOS competent E. coli BL21 (DE3) (NIPPON GENE CO., LTD.)) according to the manual, and various recombinant E. coli strains retaining a plasmid vector into which a GBA gene or an altered GBA gene was inserted were constructed.

[Synthesis Method of Protein by Recombinant E. coli, Comparative Evaluation Method, and Comparative Evaluation Result]
2-1. Synthesis of GBA Protein by Recombinant E. coli

A GBA protein or a recombinant GBA protein was synthesized using the recombinant E. coli constructed in the above 1-4.

Specifically, first, a single colony grown on an LB agar medium (containing ampicillin at a concentration of 100 mg/L) was inoculated into 4 mL of an LB liquid medium (containing ampicillin at a concentration of 100 mg/L) in a test tube, and shaken and cultured at 300 rpm and 30° C. overnight to obtain a preculture solution.

The preculture solution (2 mL) was inoculated into 50 mL of the medium for main culture (see Table 3 below for the composition) in a Sakaguchi flask, and shaken and cultured at 120 rpm and 30° C. for 72 hours to perform main culture.

TABLE 6
(Table 3) Medium for main culture

| | | |
|---|---|---|
| Glycerol | 40 | g/L |
| (NH₄)₂SO₄ | 10 | g/L |
| KH₂PO₄ | 2 | g/L |
| K₂HPO₄ | 6 | g/L |
| Yeast extract | 40 | g/L |
| MgSO₄ · 7H₂O | 1 | g |
| ADEKANOL | 0.5 | mL/L |
| Ampicillin | 100 | mg/L |
| IPTG | 100 | μM |

The culture solution after the main culture was centrifuged at 6,000×g and 4° C. for 10 minutes, the supernatant was discarded, and then a precipitate was suspended using buffer A (see Table 4 below for the composition). Thereafter, the resultant solution was centrifuged again at 6,000×g and 4° C. for 10 minutes, and the supernatant was discarded to obtain a precipitate of recombinant E. coli (followed by cryopreservation at −80° C.).

TABLE 7
(Table 4) Buffer A

| | | |
|---|---|---|
| D-mannitol | 32 | g/L |
| Trisodium citrate dihydrate | 16.54 | g/L |
| Citrate monohydrate | 1.28 | g/L |
| Tween 80 | 140 | mg/L |

### 2-2. Crushing Treatment of Bacterial Cells

The recombinant E. coli obtained in the above 2-1 was suspended in buffer A, the turbidity (OD660) was measured, and then dilution with buffer A was performed so that OD660 was 10. Zirconia silica beads (0.6 mm) were added to this suspension, and the mixture was shaken at 1300 rpm for 5 minutes by a bead-based cell disruptor (Shake Master Neo ver 1.0 manufactured by Bio Medical Science Inc.) while being cooled using an aluminum block cooled on ice, and then further cooled with an aluminum block for 5 minutes. This operation was repeated six times in total, and the cells of the bacterial cells were subjected to a crushing treatment.

Next, the resultant solution was centrifuged at 6,000×g and 4° C. for 15 minutes, and a precipitate (insoluble fraction) was collected. Each of the following solutions (1) to (4) (200 μL) was sequentially subjected to suspension and then a centrifugation treatment at 6000×g for 2 minutes twice for the collected insoluble fraction to obtain an insoluble protein.
(1) Buffer A
(2) 0.05 w/v % sodium deoxycholate (DOC·Na)-added buffer A
(3) 1 w/v % Triton X-100-added buffer A
(4) Buffer A (pH 6)

### 2-3. Denaturation (Solubilization) Treatment

Subsequently, the insoluble protein obtained by the centrifugation treatment was suspended in a 20 mM potassium phosphate buffer (pH 8) to which 6 M guanidine hydrochloride, 0.014 w/v % Tween 80, and 40 mM dithiothreitol (DTT) were added, and then allowed to stand still at 25° C. for 2 hours for incubation (denaturation (solubilization) treatment).

Next, the mixture was centrifuged at 6,000×g and 4° C. for minutes, and the insoluble component was removed by collecting the supernatant. The absorbance (280 nm) of the solution was measured using a spectrophotometer, and the protein was quantified from the obtained value (A280) according to the mathematical formula of protein concentration (mg/mL)=A280/1.7. The denominator 1.7 is an absorption coefficient calculated based on amino acid sequence information.

### 2-4. Refolding Treatment

Thereafter, a solution was prepared using a 20 mM potassium phosphate buffer (pH 8) to which 6 M guanidine hydrochloride and 0.014 w/v % Tween 80 were added so as to have a protein concentration of 1 mg/mL, and then diluted 50 times with an added 20 mM potassium phosphate buffer (pH 8) to which 40 w/v % glycerol, 0.25 w/v % Tween 80, 3 mM oxidized glutathione (GSSG), and 6 mM reduced glutathione (GSH) were added.

Incubation was started by allowing the resultant solution to stand still at 25° C. from the time point of dilution, the sample was collected 7 days after the start of incubation, and the enzyme activity was measured by the following method.

### 2-5. Measurement of Enzyme Activity

The glucocerebrosidase (GBA) is an enzyme that catalyzes a reaction of hydrolyzing dehydration-condensation sites between sugar and lipid of Glc-Cer (glucocerebroside; glycolipid). Herein, the enzyme activity of the recombinant GBA protein obtained above was measured using p-nitrophenyl-β-D-glucopyranoside (pNPG), which is a synthetic substrate, as a substrate.

Specifically, first, 90 μL of 1 w/v % Triton X-100-added buffer A, 30 L of the sample (after 7 days), and 30 μL of 50 mM pNPG-added buffer A were mixed, and the mixture was incubated at 700 rpm and 37° C. for 1 hour using a thermomixer comfort (Eppendorf). Then, 150 μL of a 0.2 N NaOH solution was added, vortexed, and then centrifuged at about several thousand rpm at room temperature for several seconds.

The supernatant (200 μL) was transferred to a microplate and the absorbance (400 nm) corresponding to the reaction product (4-nitrophenol) was measured. The capacity activity (U/mL) of the recombinant GBA protein was calculated based on a calibration curve of 4-nitrophenol prepared in advance. The specific activity (U/mg) of the recombinant GBA protein was calculated by dividing the value of the capacity activity by the set protein concentration (20 mg/L). Note that “1 U” is a unit of activity that degrades pNPG by 1 μmol per minute. For a GBA protein containing an amino acid sequence of SEQ ID NO: 1 (referred to as “H495 type protein” in the present specification) produced by E. coli in the same manner as described above using the plasmid into which the GBA gene prepared in the above 1-2. was inserted, the refolding treatment and the measurement of enzyme activity were also performed in the same manner as described above. The specific activity of the H495 type protein was 1.2 U/mg.

The results of enzyme activity measurement are shown in Table 5 below. Here, the values shown in Table 5 are relative values (%) when the specific activity value of the H495 type protein is taken as 100%.

The measurement of the enzyme activity in the present specification is performed according to the above-described method unless otherwise specified.

TABLE 8
(Table 5)
[Table 9]

| | Altered | Relative | |
|---|---|---|---|
| No. | site | activity (%)* | |
| H495 type | | 100 | Reference Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | 1 | Comparative Example |
| No. [symbol] | [symbol] | 101 | Example |
| No. [symbol] | [symbol] | 1 | Comparative Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Exampla |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Comparative Example |
| No. [symbol] | [symbol] | [symbol] | Example |
| No. [symbol] | [symbol] | [symbol] | Example |
| No. [symbol] | [symbol] | [symbol] | Comparative Example |
| No. [symbol] | [symbol] | [symbol] | Example |
| No. [symbol] | [symbol] | [symbol] | Comparative Example |
| No. [symbol] | [symbol] | [symbol] | Example |
| No. [symbol] | [symbol] | [symbol] | Example |
| No. [symbol] | [symbol] | [symbol] | Example |
| No. [symbol] | [symbol] | [symbol] | Comparative Example |
| No. [symbol] | [symbol] | [symbol] | Example |
| No. [symbol] | [symbol] | 141 | Example |
| No. [symbol] | [symbol] | [symbol] | Example |
| No. [symbol] | [symbol] | [symbol] | Example |
| No. [symbol] | [symbol] | 63 | Example |
| No. [symbol] | [symbol] | [symbol] | Example |
| No. [symbol] | [symbol] | [symbol] | Example |
| No. [symbol] | [symbol] | 72 | Example |
| No. [symbol] | [symbol] | [symbol] | Example |

[symbol]

indicates data missing or illegible when filed

### DISCUSSION

From the comparison between the H495 type protein and No. 142, the comparison between No. 145 and No. 159, and the comparison between No. 147 and No. 149, the activity improving effect of F26L was found.

From the comparison between No. 145 and No. 165, the activity improving effect of F26I was found.

From the comparison between No. 18 and No. 145, the comparison between No. 27 and No. 125, the comparison between No. 184 and No. 185, and the comparison between No. 37 and No. 167, the activity improving effect of C126T was found.

From the comparison of No. 3, No. 18, and No. 27, the activity improving effect of C342S and C126S was found.

From the comparison of No. 167, No. 168, and Nos. 186 to 193, the activity improving effect of Q57C, H60C, and T63C was found.

From the comparison between No. 167 and Nos. 171 to 176, the activity improving effect of 0143C and H145C was found.

From the comparison of No. 167, Nos. 194 to 198, No. 200, No. 201, and No. 215, and the comparison of No. 178, No. 243, No. 252, No. 254, No. 257, No. 259, and No. 263, the activity improving effect of K224C and K321C was found.

It has been reported that C342 is an amino acid residue necessary for enzyme activity (THE JOURNAL OF BIOLOGICAL CHEMISTRY VOL. 281, NO. 7, pp. 4242-4253 Feb. 17, 2006). However, it was found that in the case of substitution with serine, the activity was maintained.

### [Synthesis of Sugar Chain-Modified Glucocerebrosidase Variant]

3-1. Synthesis of Active Glucocerebrosidase Variant without Sugar Chain for Sugar Chain Modification

A plasmid into which a recombinant GBA gene containing the mutations shown in Table 6 below was inserted was obtained by the same method as in the above 1-3. Thereafter, a recombinant E. coli strain retaining the plasmid was also prepared by the same method as in the above 1-4.

Each recombinant GBA protein was obtained from each recombinant E. coli strain retaining the plasmid described in Table 6 by the same method as in the above 2-1 to 2-3.

For the recombinant GBA protein of Table 6 below, the pH was adjusted to 4.5 by adding a 1 M citric acid solution to the solution (sample) after 7 days had passed after 2-4. Refolding treatment described above. Next, the mixture was filtered through a filter sterilizing filter (manufactured by Nalgen, 0.2 μm, PES), and then desalted and concentrated (about 10 times each) by Pellicon 2, Biomax, 10 kDa, 0.1 m², V-screen (Merck). The obtained concentrated solution was purified by HiTrap SP HP, 5 mL (GE Healthcare). A solution A: buffer B (see Table 8 below for the composition) and a solution B: 1 M NaCl-added buffer A were used as solutions, and an active fraction eluted at B 25% was collected. Purification was performed by HiTrap Phenyl HP, 5 mL (GE Healthcare). A solution A: buffer C (see Table 9 below for the composition) and a solution B: ethanol were used as solutions, and an active fraction eluted at B 40% was collected. The collected solution was concentrated with Amicon Ultra-15, 3 kDa (Merck), and then freeze-dried.

3-2. Sugar Chain Modification of Active Glucocerebrosidase Variant without Sugar Chain

### (Synthesis of Sugar Chain Modification Reagent)

### (1) Preparation of Di-Man-AcS-PEG2-MAL

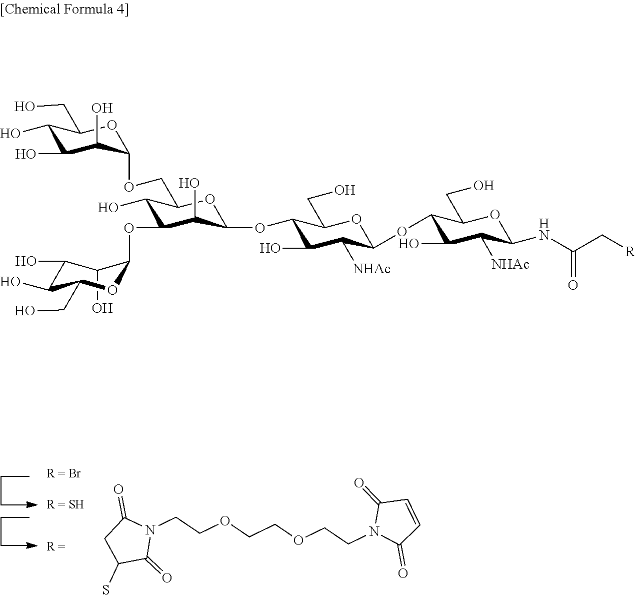

### (1-1) Preparation of Di-Man-AcSH

Di-Man-AcBr (52.5 mg, MW: 1030.8, 50.9 μmol) was dissolved in a 0.1 M phosphate buffer solution (pH=7.4, 2.0 mL), potassium thioacetate (7.0 mg, MW: 114.2, 61.1 μmol, 1.2 eq) was added thereto, and the mixture was reacted at room temperature for 1 hour. After 1 hour from the start of the reaction, cysteine (8.9 mg, MW: 121.2, 73.3 μmol, 1.2 eq of potassium thioacetate) was added. The mixture was purified by HPLC to obtain Di-Man-AcSH (38 mg, MW: 983.9, 38.6 μmol, 76%).

The purification conditions are described below.

### <Purification Conditions of Di-Man-AcSH>

Detection wavelength: 254 nm
Column: DAISOPAK SP-300-5-ODS-BIO (10×250 mm, Osaka Soda)
Column temperature: 40° C.
Mobile phase A: 0.1% TFA aqueous solution
Mobile phase B: 0.09% TFA-containing acetonitrile/water mixed solution (9:1)
Flow rate: 3.5 mL/min
Measurement time: 30 min

TABLE 10
Mobile phase gradient of HPLC condition

| Time after injection of sample | | |
|---|---|---|
| (min) | Mobile phase A (%) | Mobile phase B (%) |
| 0 | 98 | 2 |
| 30.0 | 95 | 5 |

### (1-2) Preparation of Di-Man-AcS-PEG2-MAL

Di-Man-AcSH (38 mg, MW: 983.9, 38.6 μmol, 76%) prepared in (1-1) was dissolved in a 0.1 M phosphate buffer solution (pH=6.4, 750 μL) cooled in advance, and BM (PEG) 2 (23.8 mg, MW: 308.3, 77.2 μmol, 2 eq) dissolved in DMF (188 μL) was added thereto, and the mixture was reacted at 0° C. for 30 minutes. After the reaction, the mixture was purified by HPLC to obtain Di-Man-AcS-PEG2-MAL (23.0 mg, MW: 1292.2, 17.8 μmol, 46%).

The purification conditions are described below.

### <Purification Conditions of Di-Man-AcS-PEG2-MAL>

Detection wavelength: 254 nm
Column: DAISOPAK SP-300-5-ODS-BIO (10×250 mm, Osaka Soda)
Column temperature: 40° C.
Mobile phase A: 0.1% TFA aqueous solution
Mobile phase B: 0.09% TFA-containing acetonitrile/water mixed solution (9:1)
Flow rate: 3.5 mL/min
Measurement time: 30 min

TABLE 11
Mobile phase gradient of HPLC condition

| Time after injection of sample | | |
|---|---|---|
| (min) | Mobile phase A (%) | Mobile phase B (%) |
| 0 | 95 | 5 |
| 30.0 | 85 | 15 |

### (2) Preparation of Di-Man-Asn-MAL

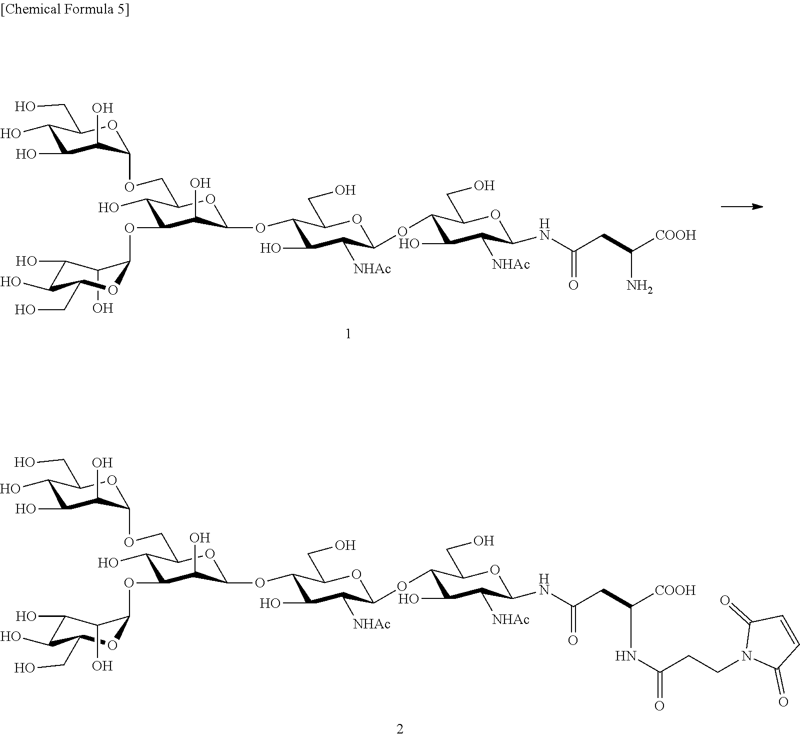

DiMan-Asn 1 (119.8 mg, MW: 1024.9, 117 μmol) was suspended in DMF, N-succinimidyl 3-maleimidopropionate (78.0 mg, MW: 266.2, 293 μmol, 2.5 eq, Wako, Product code: QA-2328) and diisopropylethylamine (DIPEA, 102 μL, 585 μmol, 5.0 eq, nacalai tesque, Product code: 14014-84) were added thereto, and the mixture was reacted at room temperature for 24 hours. After 24 hours from the start of the reaction, the reaction mixture was added dropwise to ethyl acetate and precipitated. The precipitate was washed twice with ethyl acetate and dried. The resultant product was purified by HPLC to obtain Di-Man-Asn-MAL 2 (92.0 mg, MW: 1176.05, 78.2 μmol, 67%).

The purification conditions are described below.

### <Purification Conditions of Di-Man-Asn-MAL>

Detection wavelength: 280 nm
Column: DAISOPAK SP-300-5-ODS-BIO (10×250 mm, Osaka Soda)
Column temperature: 40° C.
Mobile phase A: 0.1% THA aqueous solution
Mobile phase B: 0.09% THA-containing acetonitrile/water mixed solution (9:1)
Flow rate: 3.5 ml/min
Measurement time: 30 min

TABLE 12
Mobile phase gradient of HPLC condition

| Time after injection of sample | | |
|---|---|---|
| (min) | Mobile phase A (%) | Mobile phase B (%) |
| 0 | 98 | 2 |
| 30.0 | 94 | 6 |

### (3) Preparation of (Di-Man-C)₃-MAL

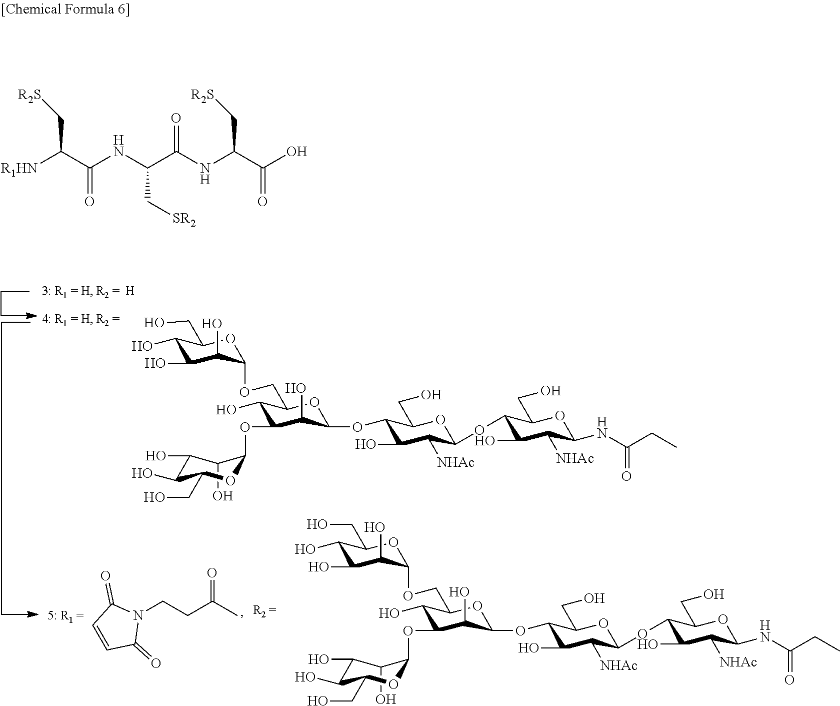

### (3-1) SPPS Step and Cutting Step

Fmoc-Cys(Trt)-OH (234 mg, 0.7 eq., 400 μmol) dissolved in DCM (3 mL) and DIPEA (0.7 eq., 400 μmol) were added to 2-chlorotrityl chloride resin (570 μmol, 1.7 mmol/g (average value of manufacturer's specification values (0.4 to 3.0 mmol/g)), and the mixture was reacted at room temperature for 1 hour. After 1 hour, the inactivation of the active chlorine on the unreacted resin was performed by washing with DCM:MeOH:DIPEA=85:10:5, followed by washing with DCM and DMF to complete the loading of the first residue amino acid (Fmoc-Cys(Trt)-OH) on the resin.

After the first residue was supported on the resin, 20% piperidine/DMF was added, and the mixture was stirred with a shaker at room temperature for 15 minutes, and then washed with DCM and DMF to complete deprotection of the Fmoc group. After washing, the Fmoc-Cys(Trt)-OH (5.0 eq., 2.0 mmol) solution preactivated by HOBt (5.0 eq., 2.0 mmol) and DIC (5.0 eq., 2.0 mmol) for 20 minutes in DMF (8 mL) in advance was added, and the mixture was stirred with a shaker at room temperature for 1 hour. Thereafter, the mixture was washed with DCM and DMF to complete the condensation of the second residue amino acid (Fmoc-Cys(Trt)-OH). The loading of Cys(Trt) of three residues on 2-chlorotriryl resin was performed by repeating the same procedure again.

After loading of Cys of three residues, the Fmoc group was deprotected and washed by the above procedure.

The resin was thoroughly washed with DCM and dried. TFA:TIPS:EDT:H₂O=92.5:2.5:2.5:2.5 (4 mL) was added to a part (150 μmol) of the dried resin, and the mixture was stirred at room temperature for 2.5 hours, then added to ice-cooled DCM, and concentrated by an evaporator. After concentration, DCM was added again and azeotroped by concentration by an evaporator. The azeotropy was repeated twice. After azeotrope, the dried and solidified peptide residue was suspended in MillQ. The suspended peptide component was washed with DCM and the aqueous layer was collected. The collected aqueous layer was freeze-dried to obtain peptide residue 3 (49.2 mg, MW: 327.4, 150 μmol, 100%).

### (3-2) Sugar Chain Modifying Step

The peptide residue 3 (29.5 mg, MW: 327.4, 90 μmol) was dissolved in 8 M Gn. HCl, 200 mM phosphate buffer solution, and 5 mM TCEP (pH=7.5, 1.5 mL). Di-Man-AcBr (278.4 mg, Mw: 1030.8, 270 μmol, 3 eq) was added to the dissolved peptide solution, the pH was adjusted to 7 with an 8 M sodium hydroxide aqueous solution, and then the solution was shaken at room temperature for 1 hour. After shaking for 1 hour, the mixture was purified by HPLC to obtain a sugar chain-modified peptide (Di-Man-C)₃ 4 (141.9 mg, MW: 3177.0, 44.7 μmol, 50%) in which three Di-Man are modified with respect to Cys of three residues.

The purification conditions are described below.

<Purification conditions of (Di-Man-C) 3>
Detection wavelength: 220 nm
Column: CAPCELLPAK UG120 (20×250 mm, Osaka Soda)
Column temperature: 40° C.
Mobile phase A: 0.1% TFA aqueous solution
Mobile phase B: 0.09% TFA-containing acetonitrile/water mixed solution (9:1)
Flow rate: 7.0 mL/min
Measurement time: 45 min

TABLE 13
Mobile phase gradient of HPLC condition

| Time after injection of sample | | |
|---|---|---|
| (min) | Mobile phase A (%) | Mobile phase B (%) |
| 0 | 99 | 1 |
| 45.0 | 84 | 16 |

### (3-3) Preparation of (Di-Man-C)₃-MAL

A sugar chain-modified peptide (Di-Man-C)₃ 4 (140.0 mg, MW: 3177.0, 44.1 μmol) was suspended in DMF, N-succinimidyl 3-maleimidopropionate (29.3 mg, MW: 266.2, 110 μmol, 2.5 eq) and DIPEA (38.5 μL, 221 μmol, 5.0 eq) were added thereto, and the mixture was reacted at room temperature for 2.5 hours. After 2.5 hours from the start of the reaction, the reaction mixture was added dropwise to ethyl acetate and precipitated. The precipitate was washed twice with ethyl acetate and dried. The dried precipitate was dissolved in Milli-Q and purified by HPLC to obtain a sugar chain-modified peptide (Di-Man-C)₃-MAL 5 (100.3 mg, MW: 3328.1, 30.1 μmol, 68%).

The purification conditions are described below.

### <Purification Conditions of (Di-Man-C)₃-MAL>

Detection wavelength: 220 nm
Column: DAISOPAK SP-300-5-ODS-BIO (10×250 mm, Osaka Soda)
Column temperature: 40° C.
Mobile phase A: 0.1% TFA aqueous solution
Mobile phase B: 0.09% TFA-containing acetonitrile/water mixed solution (9:1)
Flow rate: 3.5 mL/min
Measurement time: 30 min

TABLE 14
Mobile phase gradient of HPLC condition

| Time after injection of sample | | |
|---|---|---|
| (min) | Mobile phase A (%) | Mobile phase B (%) |
| 0 | 98 | 2 |
| 30.0 | 90 | 10 |

### (Sugar Chain Modifying Method)

A freeze-dried product of an active glucocerebrosidase variant without a sugar chain was dissolved in a 120 mM phosphate buffer (pH 6), and a 60 mM TCEP-added 120 mM phosphate buffer (pH 6) was added thereto. Incubation was performed at 4° C. for 2 hours. A sugar chain modification reagent was added, and the mixture was incubated at 4° C. for about 24 hours.

The resulting product was purified by LC-MS to obtain a sugar chain-modified active glucocerebrosidase variant. The enzyme activity measurement was performed to evaluate the progress of the modification reaction and the influence on the enzyme activity.

The results are shown in Table 6 below. For the sugar chain-modified active glucocerebrosidase variant, in the case of using Di-Man-Asn-MAL or Di-Man-AcS-PEG2-MAL as a sugar chain modification reagent, “-G1” is added to the end of the recombinant protein number, and in the case of using (Di-Man-C)₃-MAL as a sugar chain modification reagent, “-G3” is added to the end of the recombinant protein number.

TABLE 15
(Table 6)

| No. | Altered site | Residue to be modified | Modification reagent | (*1) |
|---|---|---|---|---|
| H495 type | | — | [symbol] -Asn-MAL | 510 |
| No. 44 | C342S, C126S, C248S, T61C | T61C | [symbol] -PEG₂-MAL | 500 |
| No. 54 | C342S, C126S, T61C | T61C | [symbol] -PEG₂-MAL | 500 |
| No. 48 | C342S, C126S, C248S, S148C | S148C | [symbol] -PEG₂-MAL | 500 |
| No. 50 | C342S, C126S, C248S, C-end_C | C-end_C | [symbol] -PEG₂-MAL | 500 |
| No. 51 | C342S, C126S, C248S, S242C | S242C | [symbol] -PEG₂-MAL | 500 |
| No. 168 | C342S, C248S, C126T, T61C | T61C | [symbol] -Asn-MAL | 170 |
| | | | [symbol] -MAL | 170 |
| No. 170 | C342S, C248S, C126T, Q143C | Q143C | [symbol] -Asn-MAL | 170 |
| | | | [symbol] -MAL | 170 |
| No. 178 | C342S, C248S, C126T, T61C, Q143C | T61C, Q143C | [symbol] -Asn-MAL | 170 |
| | | | [symbol] -MAL | 170 |
| No. 202 | C342S, C248S, C126T, Q143C, T61C, P58C | T61C, Q143C, P58C | [symbol] -Asn-MAL | 510 |
| | | | [symbol] -MAL | 295 |
| No. 204 | C342S, C248S, C126T, Q143C, T61C, K224C | T61C, Q143C, K224C | [symbol] -Asn-MAL | 510 |
| No. 211 | C342S, C248S, C126T, Q143C, T61C, P58C, K321C | T61C, Q143C, P58C, K321C | [symbol] -Asn-MAL | 500 |
| No. 216 | C342S, C248S, C126T, Q143C, T61C, T407C | T61C, Q143C, T407C | ( [symbol] )-MAL | 255 |
| No. 234 | C342S, C248S, C126T, Q143C, T61C, P58C | T61C, Q143C, P58C | ( [symbol] )-MAL | 255 |

| | Molecular weight | | | | | | |
|---|---|---|---|---|---|---|---|
| | [symbol] | [symbol] | [symbol] | Modification | | | |
| No. | modification | modification 1 | modification 2 | Number | (*2) | Omission of modification | |
| H495 type | 59582.5 | — | — | 0 | NT | — | (Comparative Example) |
| No. 44 | 58536.5 | 815.2 | — | 1 | 87 | No | (Example) |
| No. 54 | 55541.9 | 56823.9 | — | 1 | 89 | No | (Example) |
| No. 48 | 55534.8 | 56826.6 | — | 0, 3 | 87 | Yes | (Comparative Example) |
| No. 50 | 55624.0 | 56919.0 | — | 0, 3 | 116 | Yes | (Comparative Example) |
| No. 51 | 55834.2 | 56825.6 | — | 1 | 38 | No | (Comparative Example) |
| No. 168 | 58842.1 | 56716.9 | — | 1 | 327 | No | (Example) |
| | | 56870.7 | — | 1 | 114 | No | (Example) |
| No. 170 | 55314.8 | 56631.5 | — | 1 | 133 | No | (Example) |
| | | 58843.6 | — | 1 | 136 | No | (Example) |
| No. 178 | 55517.4 | 57563.2 | — | 2 | 153 | No | (Example) |
| | | 62173.9 | — | 2 | 136 | No | (Example) |
| No. 202 | 55523.6 | 59051.4 | — | 3 | 100 | No | (Example) |
| | | 65608.5 | — | 3 | 115 | No | (Example) |
| No. 204 | 55492.9 | 59021.0 | — | 3 | 304 | No | (Example) |
| No. 211 | 55496.9 | 60203.3 | — | 4 | 300 | No | (Example) |
| No. 216 | 56518.6 | 66508.5 | — | 3 | 96 | No | (Example) |
| No. 234 | 56536.2 | 65820.8 | 68849.8 | 3, 4 | 98 | No | (Example) |

(*1) mol equivalent (Eq) with respect to protein
(*2) Specific activity residual rate (%) after sugar chain modification treatment
NT: Not tested. [symbol]
indicates data missing or illegible when filed

It was confirmed that the free Cys residues (C126, C248, and C342) of H495 type were not modified.

In S148C and C-end_C (Cys added to the C-terminus), omission of modification was confirmed.

In S242C, a decrease in enzyme activity due to modification was confirmed.

As the modification position, T61C, P98C, Q143C, K224C, K321C, and T407C were confirmed to be suitable.

In No. 234, trace components modified at four positions (residue to be modified+C248) were detected.

### [Stability Evaluation]

Synthesis of Glucocerebrosidase Variant Added with No Sugar Chain

A plasmid into which a recombinant GBA gene containing the mutations of C248S or C248S and C342S was inserted was additionally obtained by the same method as in the above 1-3 (Nos. 19 and 42). Thereafter, a recombinant E. coli strain retaining the plasmid was also additionally prepared by the same method as in the above 1-4.

The H495 type protein and each recombinant GBA protein were obtained from each recombinant E. coli strain retaining the plasmid described in Table 7 by the same method as in the above 2-1 to 2-3.

### 4-1. Stability Evaluation in Refolding Solution

For the H495 type protein obtained above and seven recombinant GBA proteins in Table 7 below, the solution (sample) after 7 days had passed after 2-4. Refolding treatment described above was transferred to 37° C., and the transition of the residual activity was measured. The results are shown in Table 7.

TABLE 16
(Table 7)

| | | Time (h) | | | | |
|---|---|---|---|---|---|---|
| No. | Altered site | 0 | 24 | 48 | 72 | 96 |
| H495 type | — | 100 | 83 | 52 | 19 | 6 |
| No. 18 | C126S | 100 | 90 | 70 | 35 | 12 |
| No. 19 | C248S | 100 | 89 | 69 | 33 | 12 |
| No. 42 | C248S, C342S | 100 | 93 | 86 | 67 | 48 |
| No. 27 | C126S, C342S | 100 | 88 | 70 | 20 | 5 |
| No. 125 | C126T, C342S | 100 | 96 | 94 | 76 | 53 |
| No. 37 | C126S, C248S, C342S | 100 | 87 | 81 | 74 | 67 |
| No. 167 | C126T, C248S, C342S | 100 | 96 | 98 | 97 | 95 |

As shown in Table 7, it was confirmed that stability is improved by substituting a Cys residue, and stability is further improved by substituting a plurality of Cys residues, as compared with the H495 type protein.

### 4-2. Stability Evaluation 1 in Buffer

For the recombinant GBA protein of Table 10 below, the pH was adjusted to 4.5 by adding a 1 M citric acid solution to the solution (sample) after 7 days had passed after 2-4. Refolding treatment described above. Next, the mixture was filtered through a filter sterilizing filter (manufactured by Nalgen, 0.2 μm, PES), and then desalted and concentrated (about 10 times each) by Pellicon 2, Biomax, 10 kDa, 0.1 m², V-screen (Merck). The obtained concentrated solution was purified by HiTrap SP HP, 5 mL (GE Healthcare). A solution A: buffer B (see Table 8 below for the composition) and a solution B: 1 M NaCl-added buffer A were used as solutions, and an active fraction eluted at B 25% was collected. Purification was performed by HiTrap Phenyl HP, 5 mL (GE Healthcare). A solution A: buffer C (see Table 9 below for the composition) and a solution B: ethanol were used as solutions, and an active fraction eluted at B 40% was collected. The collected solution was concentrated with Amicon Ultra-15, 3 kDa (Merck), and then freeze-dried.

TABLE 17
(Table 8) Buffer B

| | | |
|---|---|---|
| D-mannitol | 32 | g/L |
| Trisodium citrate dihydrate | 16.54 | g/L |
| Citrate monohydrate | 9.95 | g/L |
| Tween 80 | 140 | mg/L |

TABLE 18
(Table 9) Buffer C

| | | |
|---|---|---|
| D-mannitol | 6.4 | g/L |
| Trisodium citrate dihydrate | 3.3 | g/L |
| Citrate monohydrate | 0.26 | g/L |
| Tween 80 | 28 | mg/L |

Cerezyme (registered trademark) and purified recombinant GBA protein (No. 176) were diluted with 0.015 w/v Tween 80-added 20 mM potassium phosphate buffer (pH 7) to 0.05 mg/mL, and incubated at 37° C., and the transition of the residual activity was measured. The results are shown in Table 10.

TABLE 19
(Table 10)

| | | Time (h) | | | | | | |
|---|---|---|---|---|---|---|---|---|
| | Altered site | 0 | [symbol] | 3 | 4 | 5.5 | 21 | 46.2 |
| Cerezyme | — | 100 | [symbol] | 53 | 47 | 40 | 6 | 0 |
| No. 176 | C126T, C246S, | 100 | 72 | [symbol] | [symbol] 5 | 61 | 40 | 23 |
| | C342S, Q143C | | | | | | | |

[symbol]

indicates data missing or illegible when filed

As shown in Table 10, it was confirmed that the recombinant GBA protein (No. 176) has improved stability with respect to Cerezyme.

### 4-3. Stability Evaluation 2 in Buffer

The recombinant GBA proteins (No. 167 and No. 178) were purified by the same method as in Stability evaluation 1 in the buffer.

Cerezyme (registered trademark) and purified recombinant GBA proteins (No. 167 and No. 178) were diluted with 0.1 w/v % Tween 80-added 50 mM potassium phosphate buffer (pH 7) to 0.01 mg/mL, and incubated at 37° C., and the transition of the residual activity was measured. The results are shown in Table 11.

TABLE 20
(Table 11)

| | | Time (h) | | | | | |
|---|---|---|---|---|---|---|---|
| | Altered site | 0 | 2 | 4 | 6 | 24 | 48 |
| Cerezyme | — | 100 | 36 | 16 | 7 | 0 | 0 |
| No. 167 | [symbol] 26T, C248S, C342S | 100 | 47 | 40 | 37 | 12 | 4 |
| No. 178 | [symbol] 26T, C248S, C347S, | 100 | 47 | 41 | 35 | 16 | 8 |
| | [symbol] C Q143 [symbol] | | | | | | |

[symbol]

indicates data missing or illegible when filed

As shown in Table 11, it was confirmed that the recombinant GBA proteins (No. 167 and No. 178) have improved stability with respect to Cerezyme.

### 4-4. Stability Evaluation 3 in Buffer

Cerezyme (registered trademark) and sugar chain-modified active glucocerebrosidase variant (No. 178-G1) were diluted with 0.015 w/v % Tween 80-added 20 mM potassium phosphate buffer (pH 7) to 0.04 mg/mL, and incubated at 37° C., and the transition of the activity was measured. The results are shown in Table 12.

TABLE 21
(Table 12)

| | | Time (h) | | | | |
|---|---|---|---|---|---|---|
| | Altered site | 0 | 2 | 4 | 6 | 24 |
| Cerezyme | — | 100 | 54 | 38 | 25 | 2 |
| No. 178-G1 | C126T, C248S, C342S, | 100 | 58 | 53 | 48 | 32 |
| | T61C, Q143C | | | | | |

As shown in Table 12, it was confirmed that the sugar chain-modified active glucocerebrosidase variant (No. 178-G1) has improved stability with respect to Cerezyme.

### 4-5. Stability Evaluation 4 in Buffer

Cerezyme (registered trademark) and sugar chain-modified active glucocerebrosidase variant (No. 178-G3) were diluted with 0.1 w/v& Tween 80-added 50 mM potassium phosphate buffer (pH 7) to 0.01 mg/ml, and incubated at 37° C., and the transition of the activity was measured. The results are shown in Table 13.

TABLE 22
(Table 13)

| | | Time (h) | | | | | |
|---|---|---|---|---|---|---|---|
| | Altered site | 0 | 2 | 4 | 6 | 24 | 48 |
| Cerezyme | — | 100 | 36 | 16 | 7 | 0 | 0 |
| No. 178-G3 | C126T, C248S, C342S, | 100 | 62 | 56 | 51 | 35 | 22 |
| | [symbol] Q [symbol] C | | | | | | |

[symbol]

indicates data missing or illegible when filed

As shown in Table 13, it was confirmed that the sugar chain-modified active glucocerebrosidase variant (No. 178-G3) has improved stability with respect to Cerezyme.

### 5. Cell Uptake Test

### Test Example 1

NR8383 cells (purchased from ATCC) were cultured in Ham's F12K medium (containing 2 mM L-glutamine, 1.5 g/L sodium bicarbonate, and 10% heat-inactivated fetal calf serum).

A test solution was prepared in a 1.5 mL tube using Ham's F12K medium so as to have the following composition. As a sample, a sugar chain-modified active glucocerebrosidase variant (No. 178-G1 or No. 178-G3) was used.

TABLE 23

| NR8383 cell | 7.5 × 10⁵ | cells |
|---|---|---|
| Sample | 0, 0.04, or 0.08 | mg/mL |
| Yeast mannan | 0 or 5 | mg/mL |
| CaCl₂ | 2.5 | mM |
| Bovine serum albumin | 0.1% | |
| Tween 80 | 0.015% | |
| Potassium phosphate | 25 | mM |

The test solution was incubated at 37° C. for 3 hours and then cooled on ice. After centrifugation (500×g, 5 min, 4° C.), the supernatant was removed, and 0.5 mL of Ham's F12K medium was added for suspension. After centrifugation (500×g, 10 min, 4° C.), the supernatant was removed, and 0.5 mL of Ham's F12K medium was added for suspension. Centrifugation (200×g, 10 min, 4° C.) was performed twice. The supernatant was removed, and 1% Triton X-100-added buffer A was added to lyse the cells. The enzyme activity and the protein concentration (660 nm Protein Assay (Pierce) were measured and the specific activity was calculated. The results are shown in Table 14.

TABLE 24
(Table 14)

| | Not added | No. 178-G1 | No. 178-G1 | No. 178-G3 | No. 178-G3 |
|---|---|---|---|---|---|
| | 0 mg/mL | 0.04 mg/mL | 0.08 mg/mL | 0.04 mg/mL | 0.08 mg/mL |
| Without yeast mannan | 157 | 228 | 228 | 215 | 239 |
| With yeast mannan | 151 | 198 | 177 | 170 | 164 |
| Difference*¹ | 6 | 30 | 51 | 45 | 75 |

*¹Difference of mannose receptor cell uptake (value of “without yeast mannan” − “with yeast mannan”)
Unit: U/g

As shown in Table 14, it was confirmed that the cell uptake amount is improved by clustering sugar chains.

### Test Example 2

The specific activity was calculated in the same manner as in Test Example 1, except that a recombinant GBA protein (No. 178) or a sugar chain-modified active glucocerebrosidase variant (No. 178-G3) was used as a sample, and a test solution was prepared so as to have the following composition. The results are shown in Table 15.

TABLE 25

| NR8383 cell | 4.5 × 10⁵ | cells |
|---|---|---|
| Sample | 0 or 0.08 | mg/mL |
| Yeast mannan | 0 or 5 | mg/mL |
| CaCl₂ | 2.5 | mM |
| Bovine serum albumin | 0.1% | |
| Tween 80 | 0.015% | |
| Potassium phosphate | 25 | mM |

TABLE 26
(Table 15)

| | Not added | No. 178 | No. 178-G3 |
|---|---|---|---|
| | 0 mg/mL | 0.08 mg/mL | 0.08 mg/mL |
| Without yeast mannan | 130 | 187 | 249 |
| With yeast mannan | 138 | 181 | 155 |
| Difference*¹ | (8) | 7 | 94 |

*¹Difference of mannose receptor cell uptake (value of “without inhibitor” − “with inhibitor”)
Unit: U/g

As shown in Table 15, it was confirmed that the uptake amount is remarkably improved by modifying sugar chains.

### Test Example 3

The specific activity was calculated in the same manner as in Test Example 1, except that a sugar chain-modified active glucocerebrosidase variant (No. 178-G3 or No. 234-G3) was used as a sample, a test solution was prepared so as to have the following composition, and incubation was performed at 37° C. for 1.5 hours. The results are shown in Table 16.

TABLE 27

| NR8383 cell | 2.8 × 10⁵ | cells |
|---|---|---|
| Sample | 0 or 0.04 | mg/mL |
| Yeast mannan | 0 or 5 | mg/mL |
| CaCl₂ | 0.5 | mM |
| Bovine serum albumin | 0.1% | |
| Tween 80 | 0.01% | |
| Potassium phosphate | 25 | mM |

TABLE 28
(Table 16)

| | Not added | No. 178-G3 | No. 234-G3 |
|---|---|---|---|
| | 0 mg/mL | 0.04 mg/mL | 0.04 mg/mL |
| Without yeast mannan | 115 | 185 | 293 |
| With yeast mannan | 123 | 133 | 159 |
| Difference*¹ | (8) | 51 | 133 |

*¹Difference of mannose receptor cell uptake (value of “without inhibitor” − “with inhibitor”)
Unit: U/g

As shown in Table 16, it was confirmed that the uptake amount is improved by adding a sugar chain modification site and not substituting C248.

The present application is based on Japanese Patent Application No. 2022-013048 filed on Jan. 31, 2022, the disclosure content of which is incorporated herein by reference in its entirety.

1. A glycosylated protein having glucocerebrosidase activity, the glycosylated protein being obtained by adding a sugar chain having a single structure to a protein added with no sugar chain and having glucocerebrosidase activity.

2. The glycosylated protein according to claim 1, wherein the sugar chain is added via a linker.

3. The glycosylated protein according to claim 2, wherein the linker includes a maleimide structure.

4. The glycosylated protein according to claim 1, wherein
the glycosylated protein has at least one sugar chain having a single structure added, and
the sugar chain having a single structure has a structure derived from a compound represented by Formula 1:

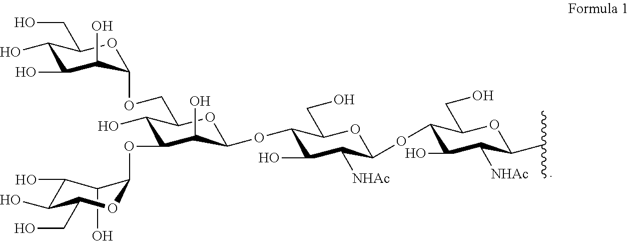

5. The glycosylated protein according to claim 1, wherein the protein added with no sugar chain and having glucocerebrosidase activity is a protein comprising at least one of amino acid substitutions below in an amino acid sequence set forth in SEQ ID NO: 1 or 2:
(1) a substitution of an amino acid at a position corresponding to position 61 of SEQ ID NO: 1 or 2 with cysteine (T61C);
(2) a substitution of an amino acid at a position corresponding to position 98 of SEQ ID NO: 1 or 2 with cysteine (P98C);
(3) a substitution of an amino acid at a position corresponding to position 143 of SEQ ID NO: 1 or 2 with cysteine (Q143C);
(4) a substitution of an amino acid at a position corresponding to position 224 of SEQ ID NO: 1 or 2 with cysteine (K224C);
(5) a substitution of an amino acid at a position corresponding to position 321 of SEQ ID NO: 1 or 2 with cysteine (K321C); and
(6) a substitution of an amino acid at a position corresponding to position 407 of SEQ ID NO: 1 or 2 with cysteine (T407C).

6. The glycosylated protein according to claim 1, wherein the sugar chain having a single structure is added to a cysteine residue constituting the protein added with no sugar chain and having glucocerebrosidase activity.

7. The glycosylated protein according to claim 1, wherein the protein added with no sugar chain and having glucocerebrosidase activity is a protein comprising at least one of amino acid substitutions below in an amino acid sequence set forth in SEQ ID NO: 1 or 2:
(7) a substitution of an amino acid at a position corresponding to position 26 of SEQ ID NO: 1 or 2 with leucine (F26L);
(8) a substitution of an amino acid at a position corresponding to position 26 of SEQ ID NO: 1 or 2 with isoleucine (F26I);
(9) a substitution of an amino acid at a position corresponding to position 126 of SEQ ID NO: 1 or 2 with threonine (C126T);
(10) a substitution of an amino acid at a position corresponding to position 126 of SEQ ID NO: 1 or 2 with serine (C126S) and a substitution of an amino acid at a position corresponding to position 342 of SEQ ID NO: 1 or 2 with serine (C342S);
(11) a substitution of an amino acid at a position corresponding to position 57 of SEQ ID NO: 1 or 2 with cysteine (Q57C);
(12) a substitution of an amino acid at a position corresponding to position 60 of SEQ ID NO: 1 or 2 with cysteine (H60C);
(13) a substitution of an amino acid at a position corresponding to position 63 of SEQ ID NO: 1 or 2 with cysteine (T63C);
(14) a substitution of an amino acid at a position corresponding to position 143 of SEQ ID NO: 1 or 2 with cysteine (Q143C);
(15) a substitution of an amino acid at a position corresponding to position 145 of SEQ ID NO: 1 or 2 with cysteine (H145C);
(16) a substitution of an amino acid at a position corresponding to position 224 of SEQ ID NO: 1 or 2 with cysteine (K224C); and
(17) a substitution of an amino acid at a position corresponding to position 321 of SEQ ID NO: 1 or 2 with cysteine (K321C).

8. The glycosylated protein according to claim 1, wherein the protein added with no sugar chain and having glucocerebrosidase activity is a protein comprising at least one of amino acid substitutions below in an amino acid sequence set forth in SEQ ID NO: 1 or 2:
(18) a substitution of an amino acid at a position corresponding to position 248 of SEQ ID NO: 1 or 2 with serine (C248S) and a substitution of an amino acid at a position corresponding to position 342 of SEQ ID NO: 1 or 2 with serine (C342S);
(19) a substitution of an amino acid at a position corresponding to position 126 of SEQ ID NO: 1 or 2 with threonine (C126T) and a substitution of an amino acid at a position corresponding to position 342 of SEQ ID NO: 1 or 2 with serine (C342S);
(20) a substitution of an amino acid at a position corresponding to position 126 of SEQ ID NO: 1 or 2 with serine (C126S), a substitution of an amino acid at a position corresponding to position 248 of SEQ ID NO: 1 or 2 with serine (C248S), and a substitution of an amino acid at a position corresponding to position 342 of SEQ ID NO: 1 or 2 with serine (C342S); and
(21) a substitution of an amino acid at a position corresponding to position 126 of SEQ ID NO: 1 or 2 with threonine (C126T), a substitution of an amino acid at a position corresponding to position 248 of SEQ ID NO: 1 or 2 with serine (C248S), and a substitution of an amino acid at a position corresponding to position 342 of SEQ ID NO: 1 or 2 with serine (C342S).

9. A composition comprising only one kind of the glycosylated protein according to claim 1.


SEQUENCE LISTING (147 sequences)

SEQ ID NO: 1 - protein, 497 aa, synthetic construct [sequence omitted]

SEQ ID NO: 2 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - protein, 497 aa, synthetic construct
  REGION 1..497: note = protein having glucocerebrosidase activity
ARPCIPKSFGYSSVVCVCNATYCDSLDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 4 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 5 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 6 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEAGVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 7 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 8 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEATVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 9 - protein, 497 aa, synthetic construct
  REGION 1..497: note = protein having glucocerebrosidase activity
ARPCIPKSFGYSSVVCVCNATYCDSIDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 10 - protein, 497 aa, synthetic construct
  REGION 1..497: note = protein having glucocerebrosidase activity
ARPCIPKSFGYSSVVCVCNATYCDSLDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 11 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 12 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 13 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 14 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 15 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFCLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 16 - protein, 497 aa, synthetic construct
  REGION 1..497: note = protein having glucocerebrosidase activity
ARPCIPKSFGYSSVVCVCNATYCDSLDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 17 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 18 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 19 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPCQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 20 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPICANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 21 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQCNHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 22 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQACHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 23 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANCTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 24 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 25 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTCTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 26 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGCGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 27 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTCLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 28 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSCPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 29 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDCQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 30 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 31 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQCHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 32 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLCNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 33 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHCFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 34 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNCSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 35 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFCLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 36 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRCVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 37 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHCLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 38 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDCEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 39 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPACATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 40 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKCWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 41 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDICKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 42 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASTDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQCNDLDAVALMHPDGSAVVVVLNRSSKDVPLTIKDPAVGFLETISPGYSIHTYLWHRQ

SEQ ID NO: 43 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 44 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQCVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 45 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPCWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 46 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWACVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 47 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLCDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 48 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPCTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 49 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSCDVPLTIKDPAVGFLETISPGYSIHTYLWHRQ

SEQ ID NO: 50 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 51 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 52 - DNA, 21 bp, synthetic construct
  misc_feature 1..21: note = primer
  source 1..21: mol_type = other DNA
ctggatccgcccacttttccc

SEQ ID NO: 53 - DNA, 18 bp, synthetic construct
  misc_feature 1..18: note = primer
  source 1..18: mol_type = other DNA
cgaatcgcagtacgtggc

SEQ ID NO: 54 - DNA, 26 bp, synthetic construct
  misc_feature 1..26: note = primer
  source 1..26: mol_type = other DNA
agcgacttttcgattcgcacctatac

SEQ ID NO: 55 - DNA, 18 bp, synthetic construct
  misc_feature 1..18: note = primer
  source 1..18: mol_type = other DNA
cgaggccattggaacgcg

SEQ ID NO: 56 - DNA, 23 bp, synthetic construct
  misc_feature 1..23: note = primer
  source 1..23: mol_type = other DNA
gacttttcgattcgcacctatac

SEQ ID NO: 57 - DNA, 21 bp, synthetic construct
  misc_feature 1..21: note = primer
  source 1..21: mol_type = other DNA
ggtcgaggccattggaacgcg

SEQ ID NO: 58 - DNA, 21 bp, synthetic construct
  misc_feature 1..21: note = primer
  source 1..21: mol_type = other DNA
gtggggagcaagttttgggag

SEQ ID NO: 59 - DNA, 25 bp, synthetic construct
  misc_feature 1..25: note = primer
  source 1..25: mol_type = other DNA
gcctgcttcgctagcaaagagcatc

SEQ ID NO: 60 - DNA, 26 bp, synthetic construct
  misc_feature 1..26: note = primer
  source 1..26: mol_type = other DNA
aagcaagcgtggggagcaagttttgg

SEQ ID NO: 61 - DNA, 27 bp, synthetic construct
  misc_feature 1..27: note = primer
  source 1..27: mol_type = other DNA
cccacgcttgcttcgctagcaaagagc

SEQ ID NO: 62 - DNA, 26 bp, synthetic construct
  misc_feature 1..26: note = primer
  source 1..26: mol_type = other DNA
aagcaaccgtggggagcaagttttgg

SEQ ID NO: 63 - DNA, 27 bp, synthetic construct
  misc_feature 1..27: note = primer
  source 1..27: mol_type = other DNA
cccacggttgcttcgctagcaaagagc

SEQ ID NO: 64 - DNA, 21 bp, synthetic construct
  misc_feature 1..21: note = primer
  source 1..21: mol_type = other DNA
attgatccgcccacttttccc

SEQ ID NO: 65 - DNA, 18 bp, synthetic construct
  misc_feature 1..18: note = primer
  source 1..18: mol_type = other DNA
cgaatcgcagtacgtggc

SEQ ID NO: 66 - DNA, 22 bp, synthetic construct
  misc_feature 1..22: note = primer
  source 1..22: mol_type = other DNA
agcctgggttttacgccggaac

SEQ ID NO: 67 - DNA, 21 bp, synthetic construct
  misc_feature 1..21: note = primer
  source 1..21: mol_type = other DNA
ttgaaacggataacccgagag

SEQ ID NO: 68 - DNA, 25 bp, synthetic construct
  misc_feature 1..25: note = primer
  source 1..25: mol_type = other DNA
tgcttaccggaagaggatacgaaac

SEQ ID NO: 69 - DNA, 24 bp, synthetic construct
  misc_feature 1..24: note = primer
  source 1..24: mol_type = other DNA
gaaattgtgcagctgaaagtcatc

SEQ ID NO: 70 - DNA, 23 bp, synthetic construct
  misc_feature 1..23: note = primer
  source 1..23: mol_type = other DNA
tgcggtactgggctgttgcttac

SEQ ID NO: 71 - DNA, 20 bp, synthetic construct
  misc_feature 1..20: note = primer
  source 1..20: mol_type = other DNA
gtgattggcctgaatagggc

SEQ ID NO: 72 - DNA, 20 bp, synthetic construct
  misc_feature 1..20: note = primer
  source 1..20: mol_type = other DNA
caggccaatcacaccggtac

SEQ ID NO: 73 - DNA, 22 bp, synthetic construct
  misc_feature 1..22: note = primer
  source 1..22: mol_type = other DNA
gcaagggcccatagacagttcc

SEQ ID NO: 74 - DNA, 19 bp, synthetic construct
  misc_feature 1..19: note = primer
  source 1..19: mol_type = other DNA
gccaatcacaccggtactg

SEQ ID NO: 75 - DNA, 24 bp, synthetic construct
  misc_feature 1..24: note = primer
  source 1..24: mol_type = other DNA
gcaaatagggcccatagacagttc

SEQ ID NO: 76 - DNA, 18 bp, synthetic construct
  misc_feature 1..18: note = primer
  source 1..18: mol_type = other DNA
aatcacaccggtactggg

SEQ ID NO: 77 - DNA, 22 bp, synthetic construct
  misc_feature 1..22: note = primer
  source 1..22: mol_type = other DNA
gcactgaatagggcccatagac

SEQ ID NO: 78 - DNA, 26 bp, synthetic construct
  misc_feature 1..26: note = primer
  source 1..26: mol_type = other DNA
aggcctgccacaccggtactgggctg

SEQ ID NO: 79 - DNA, 26 bp, synthetic construct
  misc_feature 1..26: note = primer
  source 1..26: mol_type = other DNA
gagaagcagtgcagctgaaagtcatc

SEQ ID NO: 80 - DNA, 18 bp, synthetic construct
  misc_feature 1..18: note = primer
  source 1..18: mol_type = other DNA
accggtactgggctgttg

SEQ ID NO: 81 - DNA, 21 bp, synthetic construct
  misc_feature 1..21: note = primer
  source 1..21: mol_type = other DNA
gcaattggcctgaatagggcc

SEQ ID NO: 82 - DNA, 23 bp, synthetic construct
  misc_feature 1..23: note = primer
  source 1..23: mol_type = other DNA
tgcggtactgggctgttgcttac

SEQ ID NO: 83 - DNA, 20 bp, synthetic construct
  misc_feature 1..20: note = primer
  source 1..20: mol_type = other DNA
gtgattggcctgaatagggc

SEQ ID NO: 84 - DNA, 18 bp, synthetic construct
  misc_feature 1..18: note = primer
  source 1..18: mol_type = other DNA
actgggctgttgcttacc

SEQ ID NO: 85 - DNA, 23 bp, synthetic construct
  misc_feature 1..23: note = primer
  source 1..23: mol_type = other DNA
gcaggtgtgattggcctgaatag

SEQ ID NO: 86 - DNA, 18 bp, synthetic construct
  misc_feature 1..18: note = primer
  source 1..18: mol_type = other DNA
gggctgttgcttaccctc

SEQ ID NO: 87 - DNA, 21 bp, synthetic construct
  misc_feature 1..21: note = primer
  source 1..21: mol_type = other DNA
gcaaccggtgtgattggcctg

SEQ ID NO: 88 - DNA, 19 bp, synthetic construct
  misc_feature 1..19: note = primer
  source 1..19: mol_type = other DNA
ctgttgcttaccctccaac

SEQ ID NO: 89 - DNA, 21 bp, synthetic construct
  misc_feature 1..21: note = primer
  source 1..21: mol_type = other DNA
gcaagtaccggtgtgattggc

SEQ ID NO: 90 - DNA, 22 bp, synthetic construct
  misc_feature 1..22: note = primer
  source 1..22: mol_type = other DNA
tgcctgggttttacgccggaac

SEQ ID NO: 91 - DNA, 21 bp, synthetic construct
  misc_feature 1..21: note = primer
  source 1..21: mol_type = other DNA
ttgaaacggataacccgagag

SEQ ID NO: 92 - DNA, 22 bp, synthetic construct
  misc_feature 1..22: note = primer
  source 1..22: mol_type = other DNA
cagctgcacaatttctcgttac

SEQ ID NO: 93 - DNA, 21 bp, synthetic construct
  misc_feature 1..21: note = primer
  source 1..21: mol_type = other DNA
gcagtcatccggcgtatccgc

SEQ ID NO: 94 - DNA, 20 bp, synthetic construct
  misc_feature 1..20: note = primer
  source 1..20: mol_type = other DNA
ctgcacaatttctcgttacc

SEQ ID NO: 95 - DNA, 21 bp, synthetic construct
  misc_feature 1..21: note = primer
  source 1..21: mol_type = other DNA
gcaaaagtcatccggcgtatc

SEQ ID NO: 96 - DNA, 22 bp, synthetic construct
  misc_feature 1..22: note = primer
  source 1..22: mol_type = other DNA
cacaatttctcgttaccggaag

SEQ ID NO: 97 - DNA, 24 bp, synthetic construct
  misc_feature 1..24: note = primer
  source 1..24: mol_type = other DNA
gcactgaaagtcatccggcgtatc

SEQ ID NO: 98 - DNA, 22 bp, synthetic construct
  misc_feature 1..22: note = primer
  source 1..22: mol_type = other DNA
aatttctcgttaccggaagagg

SEQ ID NO: 99 - DNA, 22 bp, synthetic construct
  misc_feature 1..22: note = primer
  source 1..22: mol_type = other DNA
gcacagctgaaagtcatccggc

SEQ ID NO: 100 - DNA, 26 bp, synthetic construct
  misc_feature 1..26: note = primer
  source 1..26: mol_type = other DNA
tgcactgcttctcgttaccggaagag

SEQ ID NO: 101 - DNA, 28 bp, synthetic construct
  misc_feature 1..28: note = primer
  source 1..28: mol_type = other DNA
gtgtggcaggcctgaatagggcccatag

SEQ ID NO: 102 - DNA, 21 bp, synthetic construct
  misc_feature 1..21: note = primer
  source 1..21: mol_type = other DNA
tcgttaccggaagaggatacg

SEQ ID NO: 103 - DNA, 24 bp, synthetic construct
  misc_feature 1..24: note = primer
  source 1..24: mol_type = other DNA
gcaattgtgcagctgaaagtcatc

SEQ ID NO: 104 - DNA, 25 bp, synthetic construct
  misc_feature 1..25: note = primer
  source 1..25: mol_type = other DNA
tgcttaccggaagaggatacgaaac

SEQ ID NO: 105 - DNA, 24 bp, synthetic construct
  misc_feature 1..24: note = primer
  source 1..24: mol_type = other DNA
gaaattgtgcagctgaaagtcatc

SEQ ID NO: 106 - DNA, 19 bp, synthetic construct
  misc_feature 1..19: note = primer
  source 1..19: mol_type = other DNA
gtgtccctgctggcttcac

SEQ ID NO: 107 - DNA, 22 bp, synthetic construct
  misc_feature 1..22: note = primer
  source 1..22: mol_type = other DNA
gcaacgctgtgccaattgtaac

SEQ ID NO: 108 - DNA, 18 bp, synthetic construct
  misc_feature 1..18: note = primer
  source 1..18: mol_type = other DNA
ctgcagttttgggccgtg

SEQ ID NO: 109 - DNA, 21 bp, synthetic construct
  misc_feature 1..21: note = primer
  source 1..21: mol_type = other DNA
gcaatgctcagcatacgcatc

SEQ ID NO: 110 - DNA, 18 bp, synthetic construct
  misc_feature 1..18: note = primer
  source 1..18: mol_type = other DNA
gaagctgcgaaatatgtg

SEQ ID NO: 111 - DNA, 22 bp, synthetic construct
  misc_feature 1..22: note = primer
  source 1..22: mol_type = other DNA
gcaatcggttaacaccactttg

SEQ ID NO: 112 - DNA, 19 bp, synthetic construct
  misc_feature 1..19: note = primer
  source 1..19: mol_type = other DNA
gccaccttgggagaaactc

SEQ ID NO: 113 - DNA, 21 bp, synthetic construct
  misc_feature 1..21: note = primer
  source 1..21: mol_type = other DNA
gcaggcaggcgccagaaaatc

SEQ ID NO: 114 - DNA, 18 bp, synthetic construct
  misc_feature 1..18: note = primer
  source 1..18: mol_type = other DNA
ctgggagcaaagcgtccg

SEQ ID NO: 115 - DNA, 19 bp, synthetic construct
  misc_feature 1..19: note = primer
  source 1..19: mol_type = other DNA
cacttgctccccacgcttg

SEQ ID NO: 116 - DNA, 22 bp, synthetic construct
  misc_feature 1..22: note = primer
  source 1..22: mol_type = other DNA
aaggatacgttctacaaacagc

SEQ ID NO: 117 - DNA, 27 bp, synthetic construct
  misc_feature 1..27: note = primer
  source 1..27: mol_type = other DNA
gcagatatcgacaatgatcggagaatc

SEQ ID NO: 118 - DNA, 20 bp, synthetic construct
  misc_feature 1..20: note = primer
  source 1..20: mol_type = other DNA
aacgacctagatgcagtagc

SEQ ID NO: 119 - DNA, 21 bp, synthetic construct
  misc_feature 1..21: note = primer
  source 1..21: mol_type = other DNA
gcactgggaggctaccaggcc

SEQ ID NO: 120 - DNA, 18 bp, synthetic construct
  misc_feature 1..18: note = primer
  source 1..18: mol_type = other DNA
gtgaaaggctttggcggc

SEQ ID NO: 121 - DNA, 25 bp, synthetic construct
  misc_feature 1..25: note = primer
  source 1..25: mol_type = other DNA
gcactgaaatttctgctccggttgg

SEQ ID NO: 122 - DNA, 20 bp, synthetic construct
  misc_feature 1..20: note = primer
  source 1..20: mol_type = other DNA
tgggccaaagtggtgttaac

SEQ ID NO: 123 - DNA, 21 bp, synthetic construct
  misc_feature 1..21: note = primer
  source 1..21: mol_type = other DNA
gcacggaaggagcaaccgttg

SEQ ID NO: 124 - DNA, 22 bp, synthetic construct
  misc_feature 1..22: note = primer
  source 1..22: mol_type = other DNA
gatccagaagctgcgaaatatg

SEQ ID NO: 125 - DNA, 23 bp, synthetic construct
  misc_feature 1..23: note = primer
  source 1..23: mol_type = other DNA
gcataacaccactttggcccaat

SEQ ID NO: 126 - DNA, 20 bp, synthetic construct
  misc_feature 1..20: note = primer
  source 1..20: mol_type = other DNA
acgatgctctttgctagcga

SEQ ID NO: 127 - DNA, 24 bp, synthetic construct
  misc_feature 1..24: note = primer
  source 1..24: mol_type = other DNA
gcacggaaagaggcgatgagtttc

SEQ ID NO: 128 - DNA, 18 bp, synthetic construct
  misc_feature 1..18: note = primer
  source 1..18: mol_type = other DNA
gacgtaccgctgaccatc

SEQ ID NO: 129 - DNA, 24 bp, synthetic construct
  misc_feature 1..24: note = primer
  source 1..24: mol_type = other DNA
gcatgaactgcggtttaaaacaac

SEQ ID NO: 130 - DNA, 26 bp, synthetic construct
  misc_feature 1..26: note = primer
  source 1..26: mol_type = other DNA
cagtaaggatccgaattcgagctccg

SEQ ID NO: 131 - DNA, 59 bp, synthetic construct
  misc_feature 1..59: note = primer
  source 1..59: mol_type = other DNA
tgatgatgatggcccatggtatatctccttcttaaagttaaacaaaattatttctagag

SEQ ID NO: 132 - DNA, 81 bp, synthetic construct
  misc_feature 1..81: note = primer
  source 1..81: mol_type = other DNA
gggccatcatcatcatcatcatcatcatcatcacagcagcggccatatcgacgacgacgacaaggcgcgcccatgtatccc

SEQ ID NO: 133 - DNA, 34 bp, synthetic construct
  misc_feature 1..34: note = primer
  source 1..34: mol_type = other DNA
ttcggatccttactgacgatgccacagataggtg

SEQ ID NO: 134 - DNA, 1494 bp, synthetic construct
  misc_feature 1..1494: note = synthetic polypeptide
  source 1..1494: mol_type = other DNA
gcgcgcccatgtatcccgaaaagcttcggttatagttctgttgtctgtgtctgtaatgccacgtactgcgattcgtttgatccgcccacttttcccgcgttaggtaccttcagccgttatgagagcacacgttcaggacgtcgcatggaactgtctatgggccctattcaggccaatcacaccggtactgggctgttgcttaccctccaaccggagcagaaatttcagaaagtgaaaggctttggcggcgcaatgacggacgcagccgctcttaatattctggcgctgtctccgccggcacaaaacctgttgctgaaatcctacttcagtgaagaaggcataggctacaacatcatacgcgttccaatggcctcgtgcgacttttcgattcgcacctatacgtatgcggatacgccggatgactttcagctgcacaatttctcgttaccggaagaggatacgaaactcaagatcccgttaattcaccgcgcgttacaattggcacagcgtcctgtgtccctgctggcttcaccgtggacatccccgacatggctgaaaaccaatggagctgtcaacggcaaagggtctctgaaaggtcaaccaggcgatatctatcaccagacctgggcgaggtatttcgtgaaattcctggatgcgtatgctgagcataagctgcagttttgggccgtgacagccgaaaacgagccaagtgcaggtttgctctcgggttatccgtttcaatgcctgggttttacgccggaacatcagcgtgacttcattgcgcgtgacttgggccccactctggcgaatagtacccaccacaatgtccggctgctgatgctggacgatcaacggttgctccttccgcattgggccaaagtggtgttaaccgatccagaagctgcgaaatatgtgcatggtatcgcggttcactggtatctggattttctggcgcctgccaaagccaccttgggagaaactcatcgcctctttccgaacacgatgctctttgctagcgaagcatgcgtggggagcaagttttgggagcaaagcgtccgcttaggcagttgggatcgaggcatgcagtacagccatagcatcattaccaacctgctgtaccatgttgttggctggactgactggaacctagcccttaatccggaaggtggtccgaattgggtacgtaactttgtggattctccgatcattgtcgatatcaccaaggatacgttctacaaacagcccatgttctaccatctggggcatttttcgaagttcattcctgaagggtcacagagagttggcctggtagcctcccagaaaaacgacctagatgcagtagcgctgatgcatccagatggcagcgcagtggtggttgttttaaaccgcagttcaaaagacgtaccgctgaccatcaaagaccctgcggtaggtttccttgaaaccattagcccaggatactccattcacacctatctgtggcatcgtcagtaa

SEQ ID NO: 135 - DNA, 1497 bp, synthetic construct
  misc_feature 1..1497: note = synthetic polypeptide
  source 1..1497: mol_type = other DNA
atggcgcgcccatgtatcccgaaaagcttcggttatagttctgttgtctgtgtctgtaatgccacgtactgcgattcgtttgatccgcccacttttcccgcgttaggtaccttcagccgttatgagagcacacgttcaggacgtcgcatggaactgtctatgggccctattcaggccaatcacaccggtactgggctgttgcttaccctccaaccggagcagaaatttcagaaagtgaaaggctttggcggcgcaatgacggacgcagccgctcttaatattctggcgctgtctccgccggcacaaaacctgttgctgaaatcctacttcagtgaagaaggcataggctacaacatcatacgcgttccaatggcctcgtgcgacttttcgattcgcacctatacgtatgcggatacgccggatgactttcagctgcacaatttctcgttaccggaagaggatacgaaactcaagatcccgttaattcaccgcgcgttacaattggcacagcgtcctgtgtccctgctggcttcaccgtggacatccccgacatggctgaaaaccaatggagctgtcaacggcaaagggtctctgaaaggtcaaccaggcgatatctatcaccagacctgggcgaggtatttcgtgaaattcctggatgcgtatgctgagcataagctgcagttttgggccgtgacagccgaaaacgagccaagtgcaggtttgctctcgggttatccgtttcaatgcctgggttttacgccggaacatcagcgtgacttcattgcgcgtgacttgggccccactctggcgaatagtacccaccacaatgtccggctgctgatgctggacgatcaacggttgctccttccgcattgggccaaagtggtgttaaccgatccagaagctgcgaaatatgtgcatggtatcgcggttcactggtatctggattttctggcgcctgccaaagccaccttgggagaaactcatcgcctctttccgaacacgatgctctttgctagcgaagcatgcgtggggagcaagttttgggagcaaagcgtccgcttaggcagttgggatcgaggcatgcagtacagccatagcatcattaccaacctgctgtaccatgttgttggctggactgactggaacctagcccttaatccggaaggtggtccgaattgggtacgtaactttgtggattctccgatcattgtcgatatcaccaaggatacgttctacaaacagcccatgttctaccatctggggcatttttcgaagttcattcctgaagggtcacagagagttggcctggtagcctcccagaaaaacgacctagatgcagtagcgctgatgcatccagatggcagcgcagtggtggttgttttaaaccgcagttcaaaagacgtaccgctgaccatcaaagaccctgcggtaggtttccttgaaaccattagcccaggatactccattcacacctatctgtggcatcgtcagtaa

SEQ ID NO: 136 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 137 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 138 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFCLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 139 - protein, 498 aa, synthetic construct
  REGION 1..498: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQC

SEQ ID NO: 140 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASSDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLCGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 141 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSCPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 142 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHCLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 143 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSCPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPACATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 144 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSPPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQSLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDICKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 145 - protein, 497 aa, synthetic construct
  REGION 1..497: note = protein having glucocerebrosidase activity
ARPCIPKSFGYSSVVCVCNATYCDSFDPPTFPALGTFSRYESTRSGRRMELSMGPIQANHCGTGLLLTLQPEQKFQKVKGFGGAMTDAAALNILALSCPAQNLLLKSYFSEEGIGYNIIRVPMASTDFSIRTYTYADTPDDFCLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEASVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 146 - DNA, 24 bp, synthetic construct
  misc_feature 1..24: note = primer
  source 1..24: mol_type = other DNA
tgcggttatccgtttcaatgcctg

SEQ ID NO: 147 - DNA, 19 bp, synthetic construct
  misc_feature 1..19: note = primer
  source 1..19: mol_type = other DNA
gagcaaacctgcacttggc